(19) United States
(12) Patent Application Publication
(10) Pub. No.: US 2026/0183339 A1
(43) Pub. Date: Jul. 2, 2026

(54) COMPOSITION COMPRISING EXTRACELLULAR MATRIX DERIVED FROM SKIN KERATINOCYTES AS ACTIVE INGREDIENT FOR PREVENTING OR TREATING CANCER BY CONTROLLING TUMOR MICROENVIRONMENT

(71) Applicant: KONKUK UNIVERSITY INDUSTRIAL COOPERATION CORP, Seoul (KR)

(72) Inventors: Sun Hee DO, Hanam-si Gyeonggi-do (KR); Hyosung KIM, Seoul (KR); Young Hyun KOO, Seoul (KR)

(73) Assignee: KONKUK UNIVERSITY INDUSTRIAL COOPERATION CORP, Seoul (KR)

(21) Appl. No.: 19/551,831

(22) Filed: Feb. 27, 2026

Related U.S. Application Data
(63) Continuation of application No. PCT/KR2024/012854, filed on Aug. 28, 2024.

(30) Foreign Application Priority Data
Aug. 28, 2023 (KR) 10-2023-0112800

(51) Int. Cl. A61K 35/36; A61K 45/06; A61P 35/00

(52) U.S. Cl. CPC A61K 35/36; A61K 45/06; A61P 35/00

(57) ABSTRACT

The present disclosure relates to a composition for preventing, alleviating, or treating cancer, the composition including a skin keratinocyte-derived extracellular matrix (ECM) as an active ingredient. The composition for preventing, alleviating, or treating cancer of the present disclosure includes the keratinocyte-derived ECM as an active ingredient, thereby significantly reducing only proliferation of cancer cells without causing side effects such as cytotoxicity in normal cells such as fibroblasts constituting the dermis of normal skin. In addition, the composition for preventing, alleviating, or treating cancer of the present disclosure was observed to: expect a significant effect in targeted therapy as well as MCF7 among breast cancer cell; have an effect of suppressing the proliferation of MDA-MB-231; be able to significantly suppress proliferation of Caki-1 and Caki-2,; and significantly reduce proliferation of PANC-1.

### CROSS-REFERENCE TO RELATED APPLICATIONS

This application is a continuation of International Application No. PCT/KR2024/012854 filed on August 28, 2024, which claims priority to Korean Patent Application No. 10-2023-0112800 filed on August 28, 2023, the entire contents of which are herein incorporated by reference.

### TECHNICAL FIELD

The present disclosure relates to a composition for preventing or treating cancer by controlling tumor microenvironment, the composition including a skin keratinocyte-derived extracellular matrix as an active ingredient.

### BACKGROUND ART

As modernization accelerates, dietary habits in Korea have recently become more westernized. With rising awareness levels, modern people are handling greater workloads than before to pursue a better life, and in this regard, they are under stress from various channels. Accordingly, many side effects are occurring, such as disease outbreak patterns and causes of death exhibiting different characteristics than before. According to the statistical investigation of cause of death published by the National Statistics Office, cancer ranked first in the mortality in 2010 with 1,444 deaths per 100,000 people in Korea, and this rate continues to rise. By type, lung cancer was the most common, followed by stomach cancer, liver cancer, colorectal cancer, and pancreatic cancer.

Cancer refers to a collection of abnormal cells arising from continuous division and proliferation due to disruption of the balance between division and death of cells caused by various causes, and is also called a tumor or a neoplasm. Cancer generally occurs in about 100 different parts of the body, including organs, white blood cells, bones, lymph nodes, and the like, and develops into serious symptoms through infiltration into surrounding tissues and metastasis to other organs (WHO, 2006). Causes of cancer include environmental or external factors such as chemicals, viruses, bacteria, and ionizing radiation, as well as internal factors such as congenital genetic mutations (Klaunig & Kamendulis, Annu Rev Pharmacol Toxicol 2004, 44:239-267).

Despite having made remarkable progress in conquering such cancer through the regulation of the cell cycle and apoptosis, as well as the exploration of new targets including oncogenes and tumor suppressor genes, cancer incidence rates continue to rise. Current treatments for cancer patients rely on surgical procedures, radiation therapy, and chemotherapy involving administration of highly cytotoxic anticancer substances. Today, approximately 60 different types of anticancer drugs are in use, and with the recent expansion of knowledge regarding cancer development and the characteristics of cancer cells, research into developing new anticancer drugs is actively underway. Regarding cancer chemotherapy, many patients suffer from side effects of anticancer drugs. Particularly, administration is limited due to toxicity of anticancer drugs. Anticancer substances used in clinical trials affect not only cancer cells but also normal cells, and repeated administration can lead to side effects such as treatment failure and issues such as drug resistance. Research is underway on new types of anticancer therapeutic agents capable of overcoming the side effects of existing anticancer drugs caused by the diversity of cancer itself and the diversification of pathogenesis.

Meanwhile, a cell-derived extracellular matrix (ECM) serves as the basic environment provided for cells to survive, and is made up of protein structures and polymers and minerals supplied from body fluids. These are regarded as a group of macromolecules forming a complex network structure while surrounding the cell exterior, occupying the space between cells to determine tissue morphology and provide an environment where cells can function normally.

According to KR10-0816395, a chondrocyte-derived extracellular matrix is collected and dried to prepare an extracellular matrix membrane, which then not only replaced periosteum for cartilage regeneration or artificial collagen membrane, but also enabled reimplantation of bone dura mater and repair of skin defects. In addition, according to KR10-2003-0093009, provided are a biodegradable polymer substrate for manufacturing an artificial organ and a preparation method thereof, wherein the biodegradable polymer substrate can reduce immune rejection by culturing cells on the biodegradable polymer substrate and then removing the cells while leaving only the extracellular matrix secreted from the cells. However, these are materials that can be used in artificial organs utilizing the extracellular matrix, and there have been no cases of their use for the purpose of cancer treatment.

Accordingly, as a result of extensive efforts made by the inventors of the present disclosure to resolve the aforementioned issues, the extracellular matrix obtained from skin keratinocytes after culturing the skin keratinocytes was confirmed to be effective in suppressing the proliferation of cancer cells, thereby completing the present disclosure.

### Disclosure of Invention

### Technical Problem

One aspect is to provide a pharmaceutical composition for preventing or treating cancer, including a skin keratinocyte-derived extracellular matrix (ECM) as an active ingredient.

Another aspect is to provide a pharmaceutical composition for suppressing angiogenesis, including a skin keratinocyte-derived ECM as an active ingredient.

Another aspect is to provide a pharmaceutical composition for suppressing cancer metastasis, including a skin keratinocyte-derived ECM as an active ingredient.

Another aspect is to provide a health functional food for preventing or alleviating cancer, including a skin keratinocyte-derived ECM as an active ingredient.

Another aspect is to provide a feed composition for preventing or alleviating cancer, including a skin keratinocyte-derived ECM as an active ingredient.

Another aspect is to provide use of a composition for preventing or treating cancer, the composition including a skin keratinocyte-derived ECM as an active ingredient.

Another aspect is to provide a method of preventing or treating cancer, including administering, to a subject in need thereof, a composition including an effective amount of a skin keratinocyte-derived ECM as an effective ingredient.

Another aspect is to provide use of a composition in the manufacture of a medicine for preventing or treating cancer, the composition including a skin keratinocyte-derived ECM as an active ingredient.

Another aspect is to provide use of a composition for suppressing angiogenesis, the composition including a skin keratinocyte-derived ECM as an active ingredient.

Another aspect is to provide a method of suppressing angiogenesis, including administering, to a subject in need thereof, a composition including an effective amount of a skin keratinocyte-derived ECM as an effective ingredient.

Another aspect is to provide use of a composition in the manufacture of a medicine for suppressing angiogenesis, the composition including a skin keratinocyte-derived ECM as an active ingredient.

Another aspect is to provide use of a composition for suppressing cancer metastasis, the composition including a skin keratinocyte-derived ECM as an active ingredient.

Another aspect is to provide a method of suppressing cancer metastasis, including administering, to a subject in need thereof, a composition including an effective amount of a skin keratinocyte-derived ECM as an effective ingredient.

Another aspect is to provide use of a composition in the manufacture of a medicine for suppressing cancer metastasis, the composition including a skin keratinocyte-derived ECM as an active ingredient.

### Solution to Problem

One aspect provides a pharmaceutical composition for preventing or treating cancer, including a skin keratinocyte-derived extracellular matrix (ECM) as an active ingredient.

In the present specification, the term "extracellular matrix (ECM)" refers to a complex aggregate of biomacromolecules filling the space within or outside cells, and consists of various molecules synthesized by cells, secreted outside the cells, and accumulated in the extracellular space. For example, the molecules constituting the ECM may include, although not limited thereto, fibrous proteins such as collagen and elastin, complex proteins such as proteoglycans and glycosaminoglycans, and cell adhesion glycoproteins such as fibronectin, laminin, and vitronectin. The molecules constituting the ECM have polyvalent binding sites, and thus may aggregate with each other or compete with other molecules to form a large matrix. The ECM contains various types of proteins necessary for cell adhesion and cell signaling, and may also contain growth factors and cytokines essential for regulating stem cell differentiation.

Meanwhile, the ECM performs physical roles such as tissue support and connection, physical boundaries, force absorption, and elasticity maintenance in skeleton, teeth, tendons, skin, and the like. In addition, the ECM also performs a physiochemical role in regulating proliferation, migration, and differentiation of cells, intracellular metabolism, and cell morphology, from the outside of the cells. The ECM is also involved in development, aging, metastasis, tissue remodeling, wound healing, biological defense, and the like, and transmits cellular information into cells via integrins.

Regulation of cellular functions by the ECM is achieved through a mechanism by which cells ultimately respond to the ECM through regulation of gene expression as ECM signals are transmitted into the cells through the cell membrane.

Interactions between cells and the ECM play an important role in regulating various biological phenomena. For example, the growth and proliferation processes of normal cells require the cells to attach to the ECM and undergo spreading. Conversely, in the absence of the cell-to-ECM interactions, the expression of receptors of the ECM acts as a negative regulator of the cell growth.

The ECM may be formed even in a generally used flat cell culture method, and preferably, may be extracted before cornification proceeds following the differentiation stage.

In an embodiment, the ECM may be obtained by culturing skin keratinocytes. Specifically, the ECM may be obtained by culturing skin keratinocytes in the presence of agarose gel, collagen gel, laminin, fibronectin, or matrigel, but is not limited thereto.

In an embodiment, the ECM may be obtained by culturing skin keratinocytes and then decellularizing the skin keratinocytes. For example, the ECM derived from skin keratinocytes may be obtained by culturing skin keratinocytes in vitro and then undergoing a decellularization process. The ECM obtained following the decellularization process may be more effective in terms of anticancer efficacy.

In the present specification, the term "decellularization" refers to removing cellular components, such as a nucleus, a cell membrane, a nucleic acid, and the like, other than the ECM from a cell or a tissue.

In the present specification, the term "decellularized ECM" refers to the ECM remaining after cellular components such as a nucleus, a cell membrane, and a nucleic acid are removed from a tissue or a cell. For example, the decellularized ECM may be the one from which only the cell nucleus and the cell membrane are removed from a cell population. As such, when only the nucleus and the cell membrane are removed from a cell population, the entire ECM may be utilized. The ECM from which only the cell nucleus and the cell membrane are removed from a cell population may utilize the entire ECM components, thereby providing a more natural, biomimetic microenvironment for cells to grow and differentiation. In addition, the decellularized ECM may have a structure overall formed by bundles of nano- or micro-sized fibers in a consistent pattern.

In an embodiment, the decellularized ECM may be obtained from cells that have been cultured in vitro. For example, the decellularizing ECM may be obtained by culturing skin keratinocytes in vitro and then decellularizing a population of the cultured skin keratinocytes. When the ECM obtained by decellularizing the population of the cultured skin keratinocytes in vitro is used in the composition for preventing, alleviating, or treating cancer according to the present disclosure, the ECM may be more effective in terms of anticancer efficacy.

In addition, the ECM obtained by decellularizing the population of skin keratinocytes exhibits excellent anticancer efficacy compared to an ECM derived from skin tissue formed within a subject. Moreover, when the decellularized ECM is obtained by cells cultured in vitro, supply issues regarded as disadvantages of the tissue-derived ECM may be resolved, use of autologous cells may offer the advantage of a low risk of immune rejection.

In an embodiment, the decellularized ECM may be obtained by a method including: culturing skin keratinocytes into a cell population; performing first decellularization by adding a solution containing a surfactant to the cell population; and performing second decellularization by adding DNase and RNase A to the solution that has completed the first decellularization. The decellularized ECM of the present disclosure obtained by the aforementioned method may have inhibitory activity on the proliferation of cancer cells.

A decellularization method for obtaining the decellularized ECM may be performed by a known method or an appropriate modification thereof. The decellularization method is intended to remove cellular components other than the ECM, and may use a physical method, a chemical method, or a mixed method thereof, and specifically, a chemical method may be used. The chemical method may be a process of immersing cultured cells in a decellularization solution for a certain period of time. The decellularization solution may be an acidic solution, a basic solution, a non-ionic detergent, or an ionic detergent, but may preferably be a non-ionic detergent.

Specifically, the decellularization method may be performed by treating with one or more selected from an ionic detergent, a non-ionic detergent, a denaturant, a hypotonic solution, DNase, RNase, and ultrasonic waves, and may be performed at a temperature range of 0 to 50 °C. For example, skin keratinocytes may be cultured in a flask to form a skin keratinocyte-derived ECM, and then treated with Triton X-100, DNase, or RNase to obtain the skin keratinocyte-derived ECM.

In an embodiment, the cancer may be at least one selected from oral cancer, liver cancer, stomach cancer, colon cancer, breast cancer, lung cancer, non-small cell lung cancer, bronchial cancer, nasopharyngeal cancer, laryngeal cancer, bone cancer, non-small cell bone cancer, pancreatic cancer, skin cancer (melanoma, etc.), basal cell carcinoma, head cancer, neck cancer, skin cancer, cervical cancer, ovarian cancer, ovarian germ cell tumor, colorectal cancer, small bowel cancer, rectal cancer, fallopian tube cancer, anal cancer, endometrial cancer, vaginal carcinoma, vulvar carcinoma, Hodgkin's disease, salivary gland cancer, tongue cancer, esophageal cancer, duodenal cancer, lymphoma, bladder cancer, gallbladder cancer, endocrine cancer, thyroid cancer, parathyroid cancer, adrenal cancer, sarcoma, pseudomyxoma peritonei, urethral cancer, ureteral cancer, penile cancer, testicular cancer, prostate cancer, chronic leukemia, acute leukemia, multiple myeloma, lymphocytic lymphoma, pediatric lymphoma, renal cancer, renal pelvis cancer, ureter cancer, renal cell carcinoma, renal pelvis carcinoma, vulvar cancer, thymus cancer, central nervous system tumor, primary central nervous system lymphoma, neuroblastoma, astrocytoma, meningioma, choroidal melanoma, ampulla of Vater cancer, peritoneal cancer, small cell carcinoma, spinal cord tumor, brain stem glioma, and pituitary adenoma, may preferably be at least one solid tumor selected from breast cancer, renal cancer, pancreatic cancer, lung cancer, and prostate cancer, and may more preferably be breast cancer.

In an embodiment, the pharmaceutical composition for preventing or treating cancer, including the skin keratinocyte-derived ECM as an active ingredient, may be for animal use.

The animal refers to any animal capable of harboring cancer, and may be, for example, a mammal including a human, and more specifically, may be a rat, a mouse, a dog, a cat, a livestock, and the like, but is not limited thereto.

In an embodiment, the pharmaceutical composition for preventing or treating cancer, including the skin keratinocyte-derived ECM as an active ingredient, may suppress the proliferation of cancer cells.

In an embodiment, the composition may control a tumormicroenvironment (TME).

In the present specification, the term "tumormicroenvironment (TME)" may refer to an environment composed of tissues and cells surrounding a tumor. For example, the TME may include, although not limited thereto, tumor cells themselves, as well as surrounding blood vessels, an ECM (surrounding tissues), immune cells, inflammatory mediators, and the like.

In an embodiment, controlling the TME may involve suppressing one or more selected from the group consisting of angiogenesis, inflammatory response, cell migration, and immunosuppression within the TME. For example, controlling the TME may involve regulating the expression of genes related to angiogenesis, inflammatory response, cell migration, or immunosuppression within the TME.

In an embodiment, the pharmaceutical composition for preventing or treating cancer, including the skin keratinocyte-derived ECM as an active ingredient, may include the decellularized ECM in a concentration of 0.001 to 10.0 (w/v)%.

For example, the pharmaceutical composition may include the decellularized ECM in a concentration of 0.001 to 10.0 (w/v)%, preferably 0.01 to 5.0 (w/v)%, more preferably 0.05 to 2.0 (w/v)%, and even more preferably 0.05 to 1.0 (w/v)%. When the concentration of the decellularized ECM included in the composition of the present disclosure is within the ranges above, the advantage in that the anticancer effect of the decellularized ECM is sufficiently exhibited while not exhibiting toxicity or side effects on living organisms or cells thereof. When the amount of the decellularized ECM of the present disclosure included in the composition is below the lower limit, the anticancer effect is not observed, and when it exceeds the upper limit, safety issues may arise and it is not economical.

In an embodiment, the pharmaceutical composition may be administered intratumorally.

In the present specification, the term "administration" refers to introduction of a given substance to a subject by an appropriate method. In the present disclosure, the pharmaceutical composition may be administered orally or parenterally during clinical administration. The parenteral administration may include administration via intratumoral injection, intravenous injection, intradermal injection, subcutaneous injection, intramuscular injection, intrapulmonary injection, intranasal injection, intrarectal injection, intraperitoneal injection, intrathecal injection, intracardiac injection, intrathoracic injection, intra-arterial injection, intraosseous injection, intra-articular injection, transdermal administration, and the like, and may preferably include intratumoral administration, but is not limited thereto. For injection, a substance may be formulated in a pharmacologically suitable buffer, preferably Hank's solution, Ringer's solution, or phosphate buffered saline. Here, the skin keratinocyte-derived ECM may be processed into a patch form, a gel form, a dispersion form, and the like, and included in the pharmaceutical composition for preventing or treating cancer.

Formulations for the parenteral administration may include a sterile solution, a non-aqueous solvent, a suspension, an emulsion, and the like. As a non-aqueous solvent and a suspension solvent, propylene glycol, polyethylene glycol, vegetable oil such as olive oil, and injectable ester such as ethyl oleate may be used.

Dosage of the pharmaceutical composition, a medicine, a pharmaceutical composition for animals, or a medicine for animals may vary depending on the age, gender, and weight of a patient or an animal to be treated, and most of all, may depend on the condition of a subject to be treated, the specific category or type of disease to be treated, the route of administration, and the properties of a therapeutic agent being used. The subject may be a mammal, for example, a human, a cow, a horse, a pig, a dog, a sheep, a goat, or a cat. The subject may be the one in need of healing from cancer.

The pharmaceutical composition, a medicine, a pharmaceutical composition for animals, or a medicine for animals may be appropriately selected depending on the absorption rate of the active ingredient in the body, the excretion rate, the age and weight, gender and condition of a patient or animal to be treated, the severity of disease to be treated. In this regard, the daily dosage may be 0.0001 to 200 mg/kg, preferably 0.0001 to 100 mg/kg, more preferably 0.001 to 100 mg/kg, more preferably 0.01 to 100 mg/kg, and more preferably 0.1 to 50 mg/kg. However, the actual dosage of the active ingredient may be determined by considering various related factors such as an amount of cells in a target tissue undergoing differentiation and proliferation, a route of administration, weight of a patient, weight, age, gender, and health status of an animal, diet, administration time, administration method, excretion rate, and severity of disease, and may be administered once a day or several times a day. Therefore, the dosage does not in any way limit the scope of the present disclosure.

The pharmaceutical composition, a medicine, a pharmaceutical composition for animals, or a medicine for animals may be administered individually as preventives or therapeutics, or in combination with other therapeutics, and may be administered sequentially or simultaneously with conventional therapeutics.

In an embodiment, the pharmaceutical composition may be administered in combination with other anticancer drugs.

In the present specification, the term "combination administration" refers to any form of simultaneous or concurrent treatment using substances useful for treating at least two separate diseases. Ingredients used in combination administration may be administered simultaneously, sequentially, in reverse order, or in any order. The ingredients may be appropriately administered at different dosages, at different administration frequency, or via different routes.

In the present specification, the expression "administered simultaneously" is not particularly limited, and refers that ingredients used in combination therapy are administered substantially simultaneously, for example, as a mixture or in immediately subsequent sequences.

In the present specification, the expression "administered sequentially" is not particularly limited, and refers that the ingredients used in combination therapy are not administered simultaneously, but are administered one after another or in groups with a specific time interval between administrations. The time interval may be the same or different between administration of each ingredient used in combination therapy, and for example, may be selected from a range of 2 minutes to 96 hours, 1 day to 7 days, or 1 week, 2 weeks or 3 weeks. Generally, the time interval between administration may range from a few minutes to several hours, and for example, may be in a range of 2 minutes to 72 hours, 30 minutes to 24 hours, or 1 to 12 hours. Additional examples of the time interval may include 24 to 96 hours, 12 to 36 hours, 8 to 24 hours, and 6 to 12 hours.

The combination therapy according to the present disclosure may be defined as being able to provide synergistic effects if the efficacy, which is measured by, for example, the degree of response, the rate of response, the time regarding disease progression or the duration of survival, is therapeutically superior to the efficacy that would be obtained by administering one or others of the ingredients of the combination therapy at conventional doses. For example, the efficacy of combination therapy is considered synergistic if it is therapeutically superior to the efficacy of each of the components used alone. In particular, a synergistic effect is considered to exist if the problematic side effects are reduced and/or less frequent than when each ingredient is used an conventional doses, without adversely affecting one or more of the degree of response, the rate of response, the time regarding disease progression, and the survival data, especially without adversely affecting the duration of response.

The other anticancer drugs may include, although not limited thereto, a standard anticancer therapeutic agent, an immune checkpoint inhibitor, an immunotherapeutic agent, etc., and may also include a therapeutic composition and an adjuvant therapeutic composition for enhancing the therapeutic effect of the standard anticancer therapeutic agent. When the pharmaceutical composition of the present disclosure is administered in combination with other anticancer drugs as described above, the anticancer efficacy may be further enhanced compared to a case when the skin keratinocyte-derived ECM and other anticancer drugs are administered individually.

Another aspect is to provide a pharmaceutical composition for suppressing angiogenesis, including the skin keratinocyte-derived ECM as an active ingredient.

In the present specification, the term "angiogenesis" refers to a process of generating new blood vessels, which rarely occurs under normal biological conditions, but is a process that is necessarily accompanied during embryogenesis, corpus luteum formation, or wound healing. In general, stimulation by angiogenesis-promoting factors triggers protease-mediated degradation of the vascular basement membrane, followed by migration, proliferation, and differentiation of vascular endothelial cells to form the lumen, and this process reconstructs the blood vessel, generating new capillaries. Such an angiogenesis process plays an important role in cancer growth and metastasis. By suppressing angiogenesis, the amount of blood supplied to cancer cells may be reduced, thereby suppressing cancer growth.

The suppression of angiogenesis may be achieved by regulating the expression of genes involved in angiogenesis suppression. For example, suppression of angiogenesis within the TME may be achieved by increasing the expression of one or more genes selected from the group consisting of PTGS2, CCL2, RHOB, CYP1B1, ANGPTL4, NRP2, CXCL8, and ZC3H12A, compared to before treatment with the composition including the skin keratinocyte-derived ECM.

In an embodiment, the suppression of angiogenesis may refer to suppression of angiogenesis within the TME.

Another aspect is to provide a pharmaceutical composition for suppressing cancer metastasis, including the skin keratinocyte-derived ECM as an active ingredient.

In the present specification, the term "cancer metastasis" refers to the phenomenon in which new tumors are formed as cancer cells detach from the primary tumor tissue, invade surrounding blood vessels or lymphatic vessels, and travel through these pathways to distant sites within the body. Suppressing cancer metastasis may contribute to suppressing cancer progression by blocking migration pathways of cancer cells or preventing the formation of new tumors.

The suppression of cancer metastasis may be achieved by regulating the expression of genes closely associated with tumor growth, invasion, and metastasis within cancer cells. For example, the suppression of cancer metastasis may be achieved by increasing the expression of any one or more genes selected from the group consisting of CCL2, CSF1, RHOB, IL1B, DNER, ITGA1, CYP1B1, TGFBR3, NRP2, IL6, PLAT, NTN4, CXCL8, ITGA2, JUP, CXCL1, and SAA1 compared to before treatment with the composition including the skin keratinocyte-derived ECM.

The "pharmaceutical composition", "medicine", "pharmaceutical composition for animals", or "medicine for animals" may further include, in addition to the skin keratinocyte-derived ECM as an active ingredient, suitable carriers, excipients, and diluents commonly used in the manufacture of pharmaceutical compositions and the like.

In the present specification, the term "pharmaceutical composition" refers to a mixture including one or more active ingredients and various ingredients that deliver or assist the same for specific therapeutic, preventive, or diagnostic purposes.

The "carrier" is a compound that facilitates the addition of a compound into a cell or tissue. The "excipient" is a compound added to impart a suitable form to an active ingredient for formulation or to increase an amount for convenience of use. The "diluent" is a compound that not only stabilizes the biologically active form of a target compound, but also dilutes the compound in water that dissolves the compound.

The carrier, the excipient, and the diluent need not be specifically limited, but examples thereof include lactose, glucose, sugar, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, and the like.

In the present specification, the term "treat" may refer that cancer or the like is cured in a shortened period of time compared to natural healing. The treatment may include amelioration and/or alleviation of cancer. In addition, the treatment may refer to healing and/or recovery of symptoms caused by cancer.

In the present specification, the term "prevention" refers to a method that partially or completely delays or prevents the onset or recurrence of a disease, disability, or associated symptoms thereof, a method that prevents acquisition or reacquisition of a disease or disability, or a method that reduces the risk of acquiring a disease or disability. For example, the prevention refers to any act of suppressing or delaying the occurrence of cancer through administration of the composition according to the present disclosure.

Another aspect provides a health functional food for preventing or alleviating cancer, including a skin keratinocyte-derived ECM as an active ingredient.

Another aspect provides a health functional food for suppressing angiogenesis, including a skin keratinocyte-derived ECM as an active ingredient.

A health functional food for suppressing cancer metastasis, including a skin keratinocyte-derived ECM as an active ingredient, is provided.

The "decellularized ECM", "cancer", "angiogenesis", and "cancer metastasis" of the present disclosure have already been described above, and thus descriptions thereof are omitted to avoid excessive redundancy.

In the present specification, the term "health functional food" refers to food manufactured (including processing) in accordance with legal standards using raw materials or ingredients having functionality beneficial to the human body (Article 3, Paragraph 1 of the Health Functional Food Act). The "health functional food" may vary in terminology and scope by country, but may correspond to "dietary supplement" in the United States, "food supplement" in Europe, "health functional food" or "food for special health use (FoSHU)" in Japan, and "health food" in China. The health functional food may refer to a food prepared or processed for health supplementation purposes by using specific ingredients as raw materials or by means of extraction, concentration, purification, mixing, and the like of specific ingredients contained in food raw materials, and refers to a food designed and processed to sufficiently exert biological regulatory functions on the body, such as biodefense, regulation of biological rhythms, and prevention and recovery of disease, by using the aforementioned ingredients. The health functional food may perform functions related to prevention and alleviation of cancer.

The "health functional food composition" of the present disclosure may include, as an active ingredient, in addition to the decellularized ECM, food raw materials available as food as described in the standards and specifications for food commonly used in food manufacturing ("food code"), and food additives described in the Food Additive Code.

Types of the food are not particularly limited. Foods to which the composition according to an aspect may be added may include, for example, various foods, powders, granules, tablets, capsules, syrups, beverages, gums, teas, vitamin complexes, and health functional food products. More specifically, formulations selected from the group consisting of powders, granules, tablets, capsules, pills, gels, jellies, suspensions, emulsions, syrups, tea bags, infused teas, gums, candies, and health drinks may be included, encompassing all health functional foods in the conventional sense.

The health functional food may include a food supplementary additive that is sitologically acceptable, and may further include a carrier that are commonly used in the preparation of health functional food.

In the present disclosure, the term "sitologically acceptable" refers to exhibiting non-toxic characteristics to cells or humans exposed to the compound.

The health functional food may be formulated in one selected from the group consisting of tablets, pills, powders, granules, powders, capsules, and liquid formulations, by additionally containing one or more of carriers, diluents, excipients, and additives.

In addition to containing the above-described active ingredient, the health functional food may contain other ingredients as essential ingredients without special limitation. For example, the health functional food may contain additional ingredients, such as various flavorings or natural carbohydrates, as in regular beverages. Examples of the natural carbohydrates include: monosaccharides, such as glucose, fructose, etc.; disaccharides, such as maltose, sucrose, etc.; polysaccharides, such as common sugar including dextrin, cyclodextrin, etc.; and sugar alcohols such as xylitol, sorbitol, erythritol, etc. In addition to those described above, natural flavorings (thaumatin, stevia extracts (e.g., rebaudioside A, glycyrrhizin, etc.)), and synthetic flavorings (e.g., saccharin, aspartame, etc.) may be advantageously used as flavorings. Proportions of the natural carbohydrates may be appropriately determined by a person skilled in the art.

In addition to those described above, the health functional food according to an aspect may contain various types of nutritional supplements, vitamins, minerals (e.g., electrolytes), flavors including synthetic flavors and natural aromas, colorants, extenders (e.g., cheese, chocolate, etc.), pectic acid and salts thereof, alginic acid and salts thereof, organic acids, protective colloidal thickeners, pH regulators, stabilizers, antiseptics, glycerin, alcohols, carbonating agents used in carbonate beverages, etc. These components may be used independently or in combination, and the proportion of these additives may also be appropriately selected by those skilled in the art.

In the health functional food, an active ingredient may be added to the food as is or used in combination with other foods or food ingredients, and may be used appropriately according to conventional methods. An amount of the active ingredient being mixed may be appropriately determined according to the purpose of use (for prevention or amelioration purposes). Generally, in the preparation of food or beverage, the health functional food may be added in an amount of about 15 wt% or less, more specifically about 10 wt% or less, relative to the raw material. However, in the case of long-term intake for health and hygiene purposes or for health control purposes, the amount may be below the ranges above.

When preparing the food composition using the decellularized ECM as an active ingredient, an amount of the active ingredient does not need to be particularly limited as long as the amount is enough to exhibit anticancer efficacy, but may be, for example, preferably 0.001 to 10.0 (w/v)%, more preferably 0.01 to 5.0 (w/v)%, more preferably 0.05 to 2.0 (w/v)%, and even more preferably 0.05 to 1.0 (w/v)%.

In the health functional food composition, the active ingredient, i.e., the decellularized ECM derived from skin keratinocytes, may vary depending on the condition and weight of a consumer, presence or severity of disease, and duration of disease, but may be appropriately selected by a person skilled in the art. For example, the daily dosage may be 0.0001 to 200 mg/kg, preferably 0.0001 to 100 mg/kg, more preferably 0.001 to 100 mg/kg, more preferably 0.01 to 100 mg/kg, and more preferably 0.1 to 50 mg/kg, and the frequency of administration does not need to be particularly limited, but may be adjusted by a person skilled in the art within a range of three times a day to once a week. In case of long-term consumption for health and hygiene purposes or health control purposes, the frequency may be below the above range.

The health functional food may be provided in combination with a conventionally known health functional food for preventing or alleviating cancer or with another existing health functional food, and such a known health functional food for preventing or alleviating cancer may be a conventionally known health functional food for preventing or alleviating cancer an existing health functional food, or a newly developed health functional food.

When the health functional food contains other health functional foods having a preventive or ameliorative effect on cancer, it is important that the amounts are combined to achieve maximum effect in the least amount without side effects, which can be readily determined by a person skilled in the art.

In addition, the decellularized ECM may be added to a general food to impart anticancer functionality. Foods may be added do not need to be specifically limited, but for example, confectionery, bread or rice cakes, cocoa products or chocolates, meat or egg products, fish products, tofus or jellies, noodles, tea, coffee, beverages, special-purpose foods, fermented soybean paste products, seasoning foods, dressings, kimchi, salted seafood products, pickled foods, braised foods, alcoholic beverages, dried foods, and other foods, as exemplified in the Standards and Specifications for Foods under Article 7 of the Food Sanitation Act ("Food Code"), may be added. In addition, dairy products, processed meat products, packaged meat, and egg products as exemplified in the processing standards and specifications of ingredients with respect to livestock products under Article 4 of the Livestock Products Sanitary Control Act ("Livestock Product Code:) may be added.

Another aspect provides a feed composition for preventing or alleviating cancer, including a skin keratinocyte-derived ECM as an active ingredient.

Another aspect provides a feed composition for suppressing angiogenesis, including a skin keratinocyte-derived ECM as an active ingredient.

Another aspect provides a feed composition for suppressing cancer metastasis, including a skin keratinocyte-derived ECM as an active ingredient.

The "decellularized ECM", "cancer", "angiogenesis", "cancer metastasis", and "suppression of angiogenesis" of the present disclosure have already been described above, and thus descriptions thereof are omitted to avoid excessive redundancy.

The "feed composition" may use, as an active ingredient, a food raw material available as a food described in the Food Standards and Specifications ("Food Code"), and a food additive described in the Food Additives Code, in addition to the decellularized ECM derived from skin keratinocytes. Even if it is not a food raw material available as a food or not a food additive, raw materials falling within the scope of single feed in Appendix 1 of the "Standards and Specifications for Feed" and raw materials falling within the scope of supplementary feed in Appendix 2 may be used.

The "feed composition" may be an extractant among supplementary feeds according to the "Standards and Specifications for Feed", and may be a mixed feed including the supplementary feed.

When preparing the feed composition, an amount of the decellularized ECM does not need to be particularly limited as long as the amount is enough to exhibit anticancer efficacy, but may be, for example, preferably 0.001 to 10.0 (w/v)%, more preferably 0.01 to 5.0 (w/v)%, more preferably 0.05 to 2.0 (w/v)%, and even more preferably 0.05 to 1.0 (w/v)%.

In the feed composition, the active ingredient, i.e., the decellularized ECM derived from skin keratinocytes, may vary depending on the condition and weight of an animal consumer, presence or severity of disease, and duration of disease, but may be appropriately selected by a person skilled in the art. For example, the daily dosage may be 0.0001 to 200 mg/kg, preferably 0.0001 to 100 mg/kg, more preferably 0.001 to 100 mg/kg, more preferably 0.01 to 100 mg/kg, and more preferably 0.1 to 50 mg/kg, and the frequency of administration does not need to be particularly limited, but may be adjusted by a person skilled in the art within a range of three times a day to once a week. In case of long-term consumption for health and hygiene purposes or health control purposes, the frequency may be below the above range.

Another aspect provides a method of preventing or treating cancer, including administering, to a subject in need thereof, a composition including an effective amount of a skin keratinocyte-derived ECM as an effective ingredient.

The method of treating cancer may be to administer the composition intratumorally to a human or a non-human animal, particularly a mammal. The method may be to administer the composition intratumorally to a subject harboring cancer.

A dosage, an administration method, and frequency of administrations for the treatment may refer to the dosage, the administration method, and the frequency of administration of the pharmaceutical composition, the medicine, the pharmaceutical composition for animals, or the medicine for animals.

Another aspect provides a method of suppressing angiogenesis, including administering, to a subject in need thereof, a composition including an effective amount of a skin keratinocyte-derived ECM as an effective ingredient.

Another aspect provides a method of suppressing cancer metastasis, including administering, to a subject in need thereof, a composition including an effective amount of a skin keratinocyte-derived ECM as an effective ingredient.

Another aspect provides use of a composition in the manufacture of a medicine for preventing or treating cancer, the composition including a skin keratinocyte-derived ECM as an active ingredient.

Another aspect provides use of a composition in the manufacture of a medicine for suppressing angiogenesis, the composition including a skin keratinocyte-derived ECM as an active ingredient.

Another aspect provides use of a composition in the manufacture of a medicine for suppressing cancer metastasis, the composition including a skin keratinocyte-derived ECM as an active ingredient.

Another aspect provides use of a composition for preventing or treating cancer, the composition including a skin keratinocyte-derived ECM as an active ingredient.

Another aspect provides use of a composition for suppressing angiogenesis, the composition including a skin keratinocyte-derived ECM as an active ingredient.

Another aspect provides use of a composition for suppressing cancer metastasis, the composition including a skin keratinocyte-derived ECM as an active ingredient.

The technical terms and methods described for the present disclosure may be equally applied to the respective disclosures.

### Advantageous Effects of Disclosure

The composition for preventing, alleviating, or treating cancer of the present disclosure includes the skin keratinocyte-derived ECM, and thus may significantly reduce only the proliferation of cancer cells without causing side effects such as cytotoxicity to normal cells such as fibroblasts that constitute the dermis of normal skin.

In addition, the composition for preventing, alleviating, or treating cancer of the present disclosure was observed to: expect a significant effect in targeted therapy as well as MCF7 among breast cancer cell; have an effect of suppressing the proliferation of MDA-MB-231 which currently lacks effective treatments; be able to significantly suppress proliferation of Caki-1 and Caki-2, which are clear cell carcinomas classified as intractable cancer among renal cancer cells and known to have many distant metastasis recurrences; and significantly reduce proliferation of PANC-1, which is a pancreatic cancer cell line classified as intractable cancer that is difficult to detect early among cancer cells and is found in the late stage where surgery and treatment are limited, and shows low treatment responsiveness to the latest immunotherapy drugs and low treatment responsiveness to targeted therapy and traditional chemotherapy. In addition, the composition for preventing, alleviating, or treating cancer of the present disclosure may be applied to the treatment of various types of carcinomas and solid tumors occurring in the skin, in addition to breast cancer, renal cancer, and pancreatic cancer.

In addition, the composition for preventing, ameliorating, or treating cancer of the present disclosure may be used as a therapeutic agent in combination with other anticancer agents.

In addition, the composition for preventing, alleviating, or treating cancer of the present disclosure may be used for a treatment method of reducing the tumor size through intratumoral injection before surgery unlike traditional chemotherapy, without side effects.

### BRIEF DESCRIPTION OF DRAWINGS

FIG. 1 is a graph showing cytotoxicity of skin keratinocyte-derived extracellular matrix (HaCaT_ECM) according to an embodiment, against human dermal fibroblasts (HDFs), which constitute the dermis of normal skin, and various cancer cells (MDA-MB-231, MCF7, Caki-1, Caki-2, PANC-1, MiaPaCa-2, LNCaP, PC3, and A549). *, **, and *** each indicate the presence of significant differences at p<0.05, p<0.01, and p<0.001, as compared to treatment groups at respective concentrations (Bonferroni post hoc test).

FIG. 2 shows graphs showing cytotoxicity of an exosome derived from umbilical cord-derived mesenchymal stem cells (UC-MSCs), decellularized extracellular matrix (dECM) derived from UC-MSCs, and porcine skin-derived dECM, against human dermal fibroblast-neonatal (HDFn), Caki-2, MDA-MB-231, A549, MiaPaCa-2, and PC3.

FIGS. 3A-3C show results of RNA sequencing analysis after treating MDA-MB-231 cells with HaCaT_ECM according to an embodiment, wherein FIG. 3A shows genes that changed by at least two-fold compared to a control group that is not treated with the HaCaT_ECM, as categorized by gene type, FIG. 3B shows proportions of genes that show an increase or a decrease within each gene category, and FIG. 3C shows a scatter plot of genes showing such a two-fold or more change. In FIG. 3C, dots 310 indicate a decrease, red dots 320 indicate an increase, and gray 330 dots indicate genes that do not show a difference of at least two-fold.

FIGS. 4A-4F show results of RNA sequencing analysis regarding gene expression changes after treating MDA-MB-231 cells a with HaCaT_ECM according to an embodiment, wherein FIG. 4A shows changes in the expression of genes related to angiogenesis, FIG. 4B shows changes in the expression of genes related to inflammatory response, FIG. 4C shows changes in the expression of genes related to an extracellular membrane, FIG. 4D shows changes in the expression of genes related to cell migration, FIG. 4E shows changes in the expression of genes related to apoptosis, and FIG. 4F shows changes in the expression of genes related to immune checkpoint.

FIGS. 5A and 5B show results of DAVID functional annotation analysis on 162 genes selected at the level of fold change 2, normalized data 4 (log 2), and p-value of 0.05, based on RNA sequencing analysis of MDA-MB-231 cells treated with HaCaT_ECM according to an embodiment.

FIGS. 6A-6C show results of RNA sequencing analysis after treating MCF7 cell lines with HaCaT_ECM according to an embodiment, wherein FIG. 6A shows genes that changed by at least two-fold compared to a control group that is not treated with the HaCaT_ECM, as categorized by gene type, FIG. 6B shows proportions of genes that show an increase or a decrease within each gene category, and FIG. 6C shows a scatter plot of genes showing such a two-fold or more change. In FIG. 6C, dots 610 indicate a decrease, dots 620 indicate an increase, and dots 630 indicate genes that do not show at least a twofold difference.

FIGS. 7A-7E show results of RNA sequencing analysis regarding gene expression changes after treating MCF7 cells with HaCaT_ECM according to an embodiment, wherein FIG. 7A shows changes in the expression of genes related to DNA repair, FIG. 7B shows changes in the expression of genes related to cell cycle, FIG. 7C shows changes in the expression of genes related to apoptosis, FIG. 7D shows changes in the expression of genes related to cell aging, and FIG. 7E shows changes in the expression of RNA splicing.

FIGS. 8A and 8B show results of DAVID functional annotation analysis on 1403 genes selected at the level of fold change 2, normalized data 4 (log 2), and p-value of 0.05, based on RNA sequencing analysis of MCF7 cells treated with HaCaT_ECM according to an embodiment.

FIG. 9 shows photographs of a mouse tumor 3 weeks after administration of HaCaT_ECM according to an embodiment.

FIG. 10 shows a graph measuring changes in tumor size resulting from administration of HaCaT_ECM according to an embodiment.

FIG. 11 shows a graph measuring changes in mouse body weight following administration of HaCaT_ECM according to an embodiment.

### BEST MODE FOR THE INVENTION

Hereinafter, preferable examples are presented to help understanding the present disclosure. However, the following examples are only presented for easier understanding of the present disclosure, and the contents of the present disclosure are not limited by the following examples. Examples can undergo various modifications, and thus examples are not limited to those disclosed below and can be implemented in various forms.

### Example 1. Obtaining skin keratinocyte-derived decellularized ECM (HaCaT_ECM)

Skin keratinocytes (HaCaT cells) were cultured and subjected to a decellularization process, thereby obtaining an extracellular matrix (HaCaT_ECM).

Specifically, the HaCaT cells were seeded at a density of 2 x 10⁴ cells/cm² in a T-175 flask, and cultured in Dulbecco's modified eagle medium supplemented with 10 % fetal bovine serum and under 1% antibiotics. When cell confluency reached 80%, the cells were washed with phosphate-buffered saline (PBS), and then lysed in a mixed solution of 0.25% Triton X-100 and 10 mM NH4OH at 37°C for 90 seconds. Afterwards, DNase (e.g., deoxyribonuclease I) and RNase A, each at a concentration of 50 U/mL, were added thereto and allowed for a reaction for 90 minutes to remove the nucleic acid, and the resulting ECM was washed with PBS. The final product was freeze-dried at -80°C to obtain decellularized HaCaT_ECM.

### Experimental Example 1: Confirmation of inhibitory effect of decellularized HaCaT-ECM on cancer cell proliferation

Using CCK-8 assay, it was confirmed whether the decellularized HaCaT_ECM obtained by the method of Example 1 exhibits cytotoxicity against human dermal fibroblasts-neonatal (HDFns) and various cancer cells.

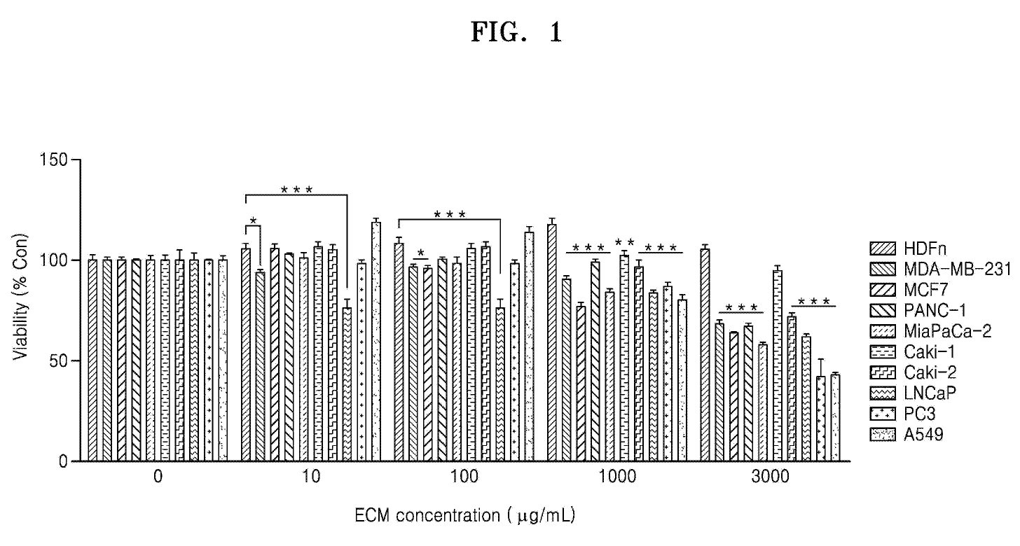

FIG. 1 is a graph showing cytotoxicity of the HaCaT_ECM according to an aspect, against human dermal fibroblasts (HDFs), which constitute the dermis of normal skin, and various cancer cells.

As shown in FIG. 1, it was specifically confirmed that the HaCaT_ECM obtained by the method of Example 1 had no effect on normal human dermal fibroblasts (e.g., HDFns) at all concentrations, but significantly inhibited the proliferation of a breast cancer cell line (MCF7) as well as a hormone receptor triple-negative breast cancer cell line (MDA-MB-231), which is a representative refractory solid cancer, pancreatic cancer cell lines (PANC-1 and MiaPaCa-2), renal cancer cell lines (Caki-1 and Caki-2), prostate cancer cell lines (LNCaP and PC3), and a lung cancer cell line (A549).

These experimental results suggest that the composition including the skin keratinocyte-derived ECM of the present disclosure as an active ingredient exhibits anticancer effects through significantly reducing the proliferation of various cancer cells without affecting normal cells, and then selectively acting on cancer cells.

### Experimental example 2. Evaluation of inhibitory effect of exosome derived from UC-MSC, decellularized ECM derived from UC-MSC, and porcine skin-derived decellularized MSC

The renal cancer cell line (Caki-2), the hormone receptor triple-negative breast cancer cell line (MDA-MB-231), the lung cancer cell line (A549), the pancreatic cancer cell line(MiaPaCa-2), and the prostate cancer cell line(PC3) were each treated with an exosome derived from umbilical cord-derived mesenchymal stem cells (UC-MSCs), a decellularized ECM derived from US-MSCs, and a porcine skin-derived decellularized ECM, at the same concentration as the decellularized ECM derived from skin keratinocytes obtained by the method of Example 1. Then, the cytotoxicity against these cell lines was confirmed using CCK-8 assay.

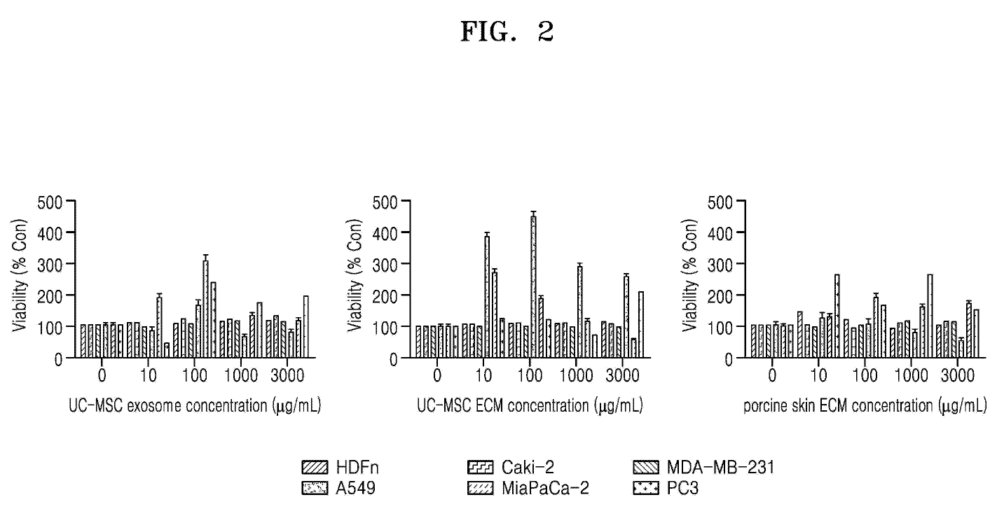

FIG. 2 shows graphs showing the cytotoxicity of the exosome derived from UC-MSCs, the decellularized ECM (dECM) derived from UC-MSCs, and the porcine skin-derived dECM, against human dermal fibroblasts-neonatal (HDFn), Caki-2, MDA-MB-231, A549, MiaPaCa-2, and PC3.

As shown in FIG. 2, it was confirmed that the renal cancer cell line (Caki-2) and the hormone receptor triple-negative breast cancer cell line (MDA-MB-231) that were treated with the exosome derived from UC-MSCs, the dECM derived from UC-MSCs, and the porcine skin-derived dECM showed cell proliferation at a level similar to that of the normal cells, HDFns, at all concentrations.

Meanwhile, it was confirmed that lung cancer cell line (A549) treated with the dECM derived from UC-MSCs at a concentration of 10 to 3000 µg/mL showed more active cell proliferation than the normal cells, HDFns.

In addition, it was confirmed that the pancreatic cancer cell line (MiaPaCa-2) treated with 10 to 100 µg/mL of the exosome derived from UC-MSCs, 10 to 100 µg/mL of the dECM derived from UC-MSCs, and 100 to 3000 µg/mL of porcine skin-derived dECM showed more active cell proliferation than the normal HDFns.

In addition, it was confirmed that the prostate cancer cell line (PC3) treated with 100 to 3000 µg/mL of the exosome derived from UC-MSCs, 10 to 100 µg/mL of dECM derived from UC-MSCs, and 10 to 3000 µg/mL of the porcine skin-derived dECM showed more active cell proliferation than the normal HDFns.

These experimental results indicate that the exosome derived from UC-MSCs, the dECM derived from UC-MSCs, and the porcine skin-derived dECM had no inhibitory effect on the proliferation of cancer cells, but were able to promote the proliferation of cancer cells.

### Experimental Example 3. Analysis of changes in gene expression of cancer cells before and after HaCaT_ECM treatment

### 3.1 Confirmation of changes in gene expression in MDA-MB-231 cells

### 3.1.1 Confirmation of gene group with significant expression changes

First, MDA-MB-231 cells, a hormone receptor triple-negative breast cancer cell line, were treated with the HaCaT_ECM obtained by the method of Example 1 at a concentration of 3000 μg/mL and cultured for 24 hours. Total RNA was extracted from the cultures by using the RNeasy Mini kit (Qiagen), and the concentration was measured using NanoVue (GE HealthCare, USA). Subsequently, mRNA sequencing was requested (Ebiogen, Republic of Korea).

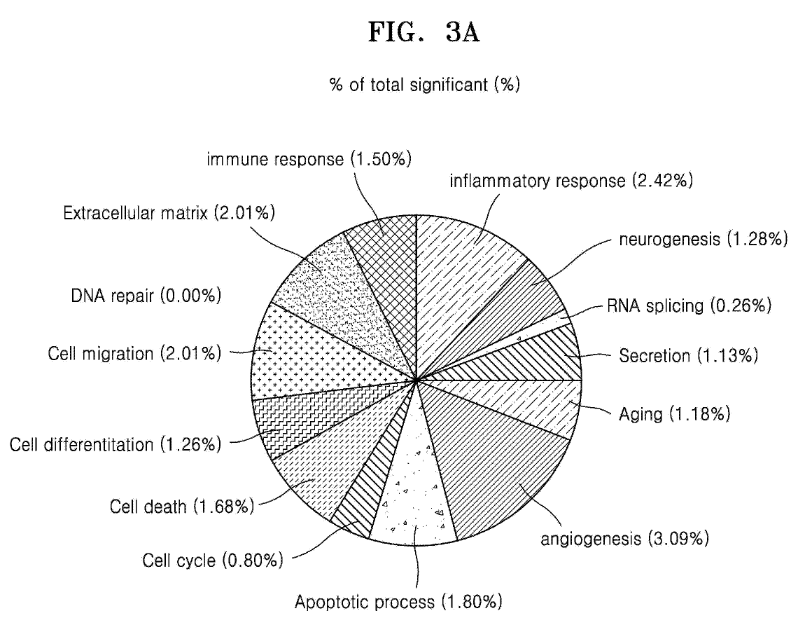

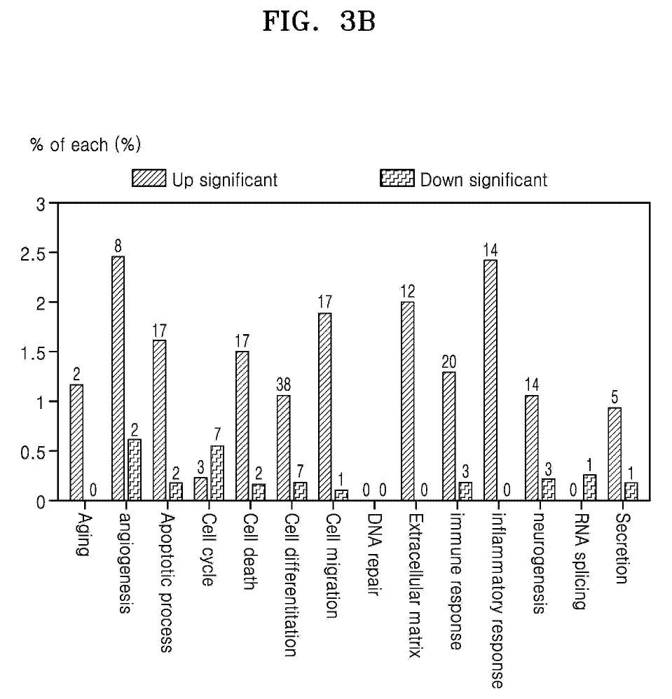

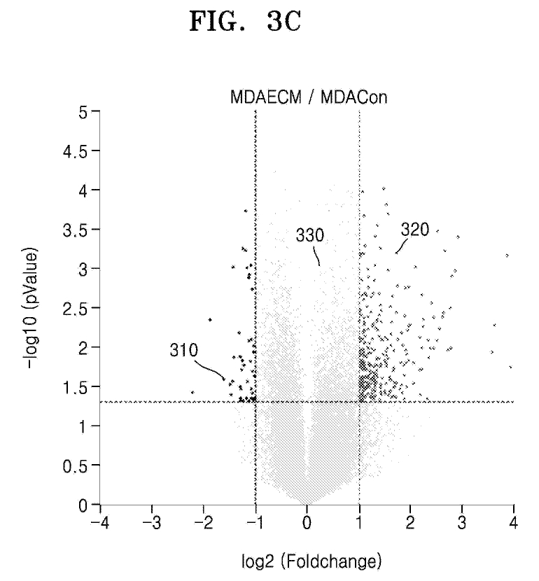

FIG. 3 shows the results of RNA sequencing analysis after treating MDA-MB-231 cells with the HaCaT_ECM according to an embodiment. FIG. 3A shows genes that changed by at least two-fold compared to a control group that is not treated with the HaCaT_ECM, as categorized by gene type, FIG. 3B shows proportions of genes that show an increase or a decrease within each gene category, and FIG. 3C shows a scatter plot of genes showing such a two-fold or more change. In FIG. 3C, dots 310 indicate a decrease, dots 320 indicate an increase, and dots 330 indicate genes that do not show a difference of at least two-fold.

As shown in FIG. 3, it was confirmed that the MDA-MB-231 cells treated with the HaCaT_ECM according to an embodiment of the present disclosure showed significant changes in the expression of genes related to angiogenesis, inflammatory response, cell migration, apoptosis (apoptotic process or cell death), and immune response (showing at least a two-fold difference in the expression levels compared to before the HaCaT_ECM treatment).

### 3.1.2 Confirmation of expression changes in specific genes

MDA-MB-231 cells, a hormone receptor triple-negative breast cancer cell line, were treated with the HaCaT_ECM obtained by the method of Example 1, and changes in the expression of genes were specifically observed through RNA sequencing analysis.

### Genes related to angiogenesis

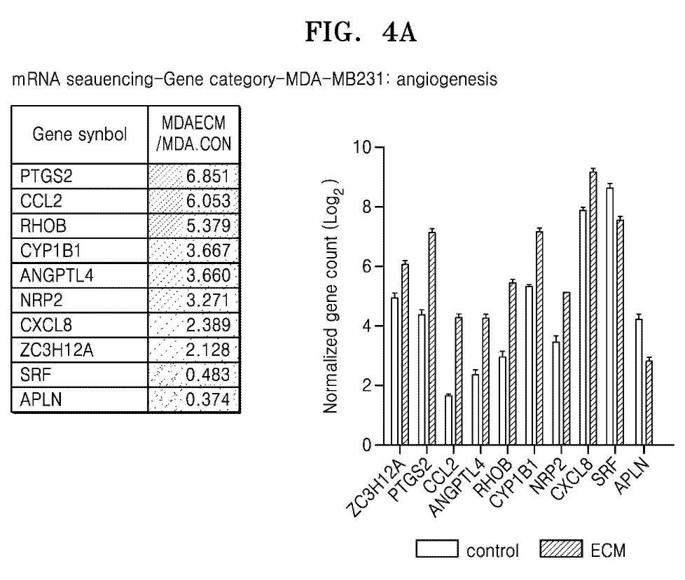

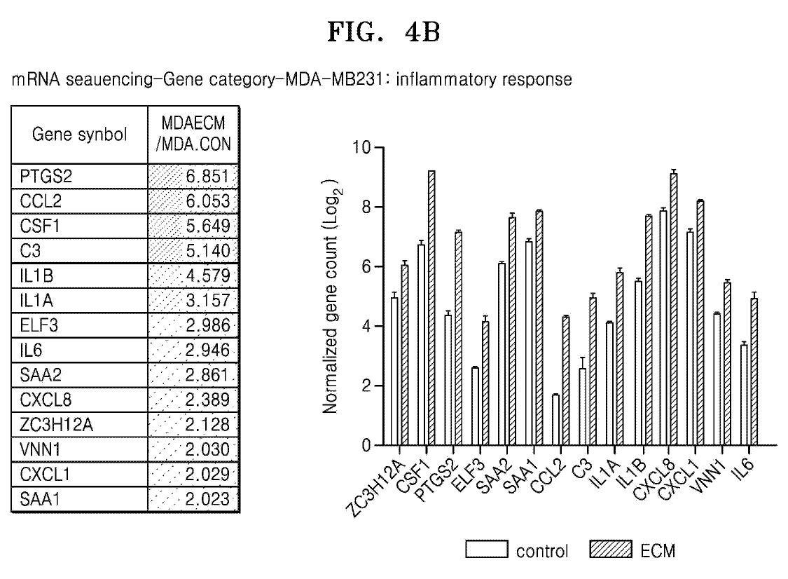

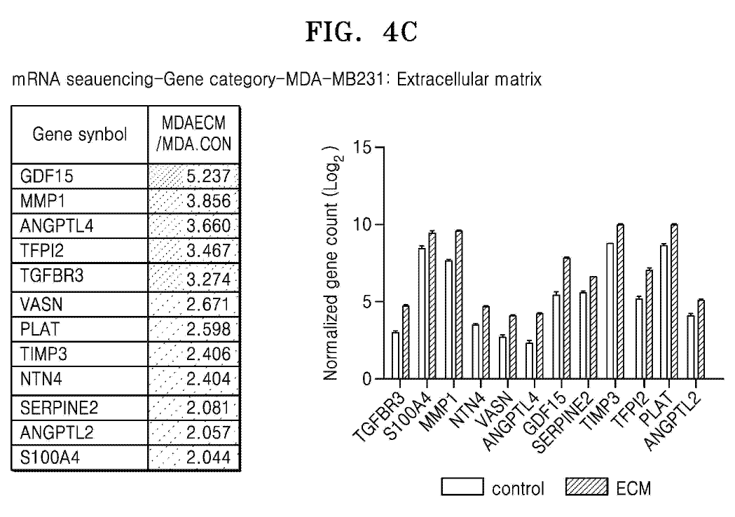

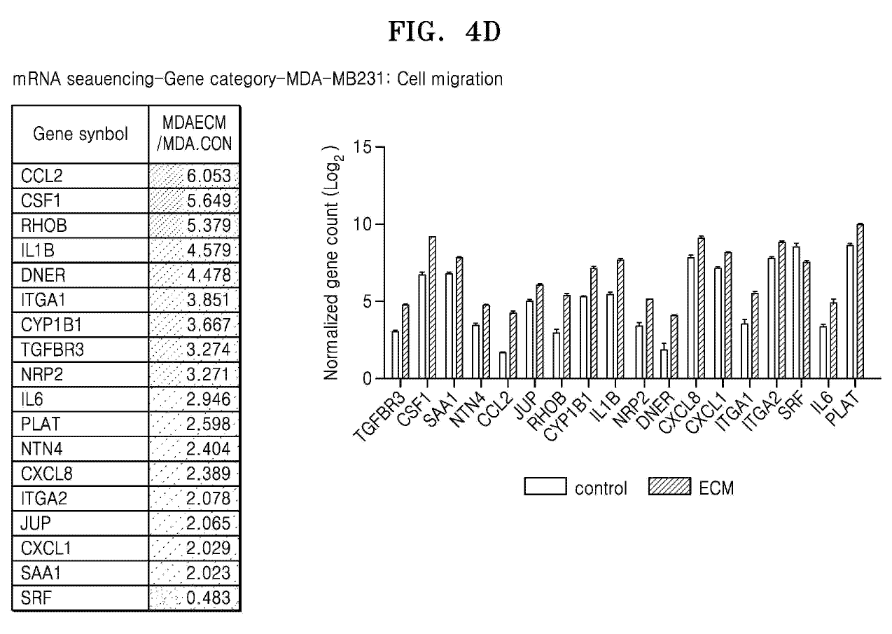

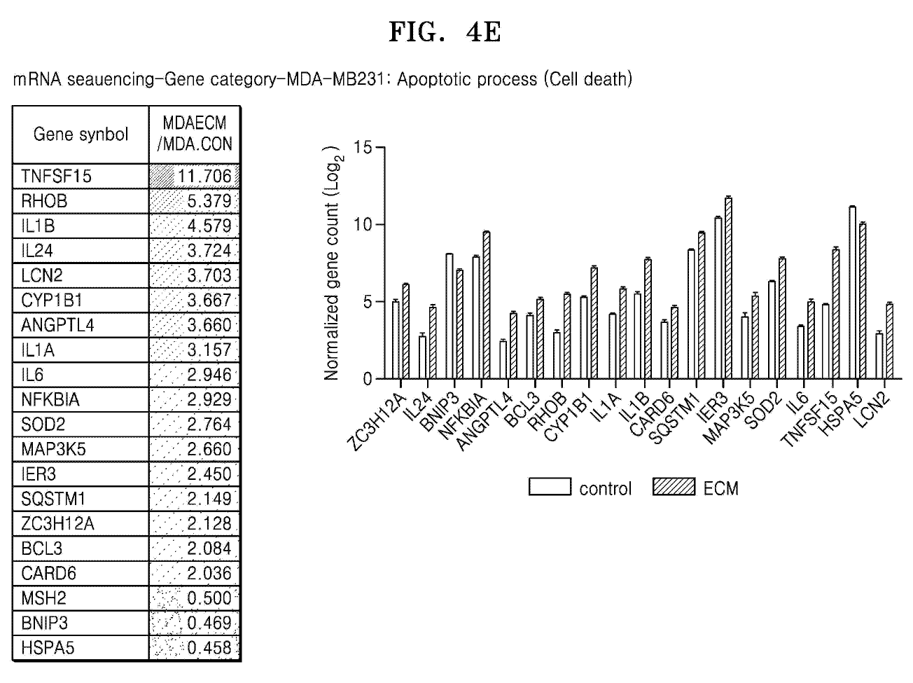

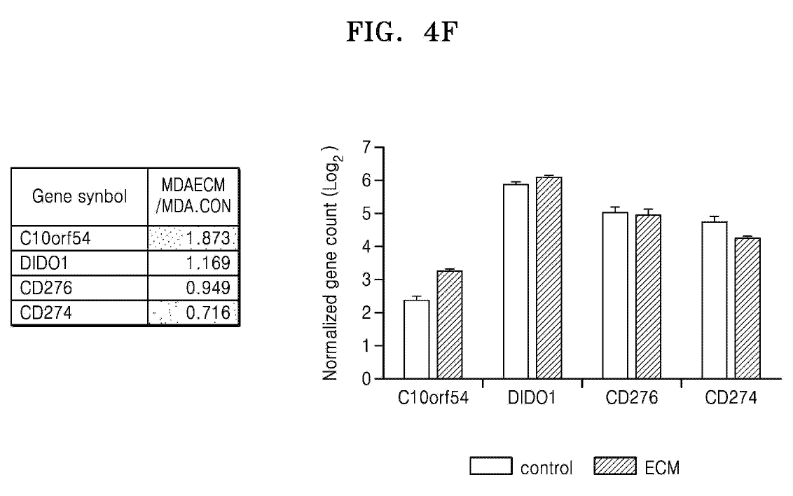

FIG. 4A shows the results of analyzing the changes in the expression of genes related to angiogenesis in the MDA-MB-231 cells treated with the HaCaT_ECM of Example 1.

As shown in FIG. 4A, when the MDA-MB-231 cells were treated with the HaCaT-ECM, the expression of PTGS2, CCL2, RHOB, CYP1B1, ANGPTL4, NRP2, CXCL8, and ZC3H12A genes, which are genes related to angiogenesis, was increased by at least two-fold compared to before the HaCaT_ECM treatment.

The genes of which the expression was increased by at least two-fold compared to before the HaCaT_ECM treatment are those related to angiogenesis in a tumor microenvironment (TME). In particular, PTGS2 is an enzyme involved in prostaglandin (PG) synthesis, which promotes angiogenesis and affects tumor growth and metastasis, and CCL2 is a chemokine that induces inflammatory responses associated with angiogenesis in the TME and also affects ECM remodeling. RHOB is a gene belonging to the Rho GTPase family, involved in regulating angiogenesis through interactions between tumor cells and the ECM, and CYP1B1 participates in promoting inflammation and angiogenesis by generating reactive oxygen species (ROS) in response to various environmental stimuli.

These experimental results indicate that the composition including the skin keratinocyte-derived ECM as an active ingredient according to the present disclosure exhibits anticancer effects through not only suppressing the proliferation of tumor cells, but also controlling the TME by regulating the expression of genes related to angiogenesis.

### Genes related to inflammatory response

FIG. 4B shows the results of analyzing the changes in the expression of genes related to inflammatory response in the MDA-MB-231 cells treated with the HaCaT_ECM of Example 1.

As shown in FIG. 4B, when the MDA-MB-231 cells were treated with the HaCaT_ECM, the expression of PTGS2, CCL2, CSF1, C3, IL1B, IL1A, ELF3, IL6, SAA2, CXCL8, ZC3H12A, VNN1, CXCL1, and SAA1 genes, which are genes related to inflammatory response, was increased by at least two-fold compared to before the HaCaT_ECM treatment.

The genes of which the expression was increased by at least two-fold compared to before the HaCaT_ECM treatment are those related to inflammatory response in the TME. In particular, PTGS2 (COX-2) is involved in inflammatory response, pain, fever, and angiogenesis promotion, and CCL2 is a chemokine involved in inflammatory response of tumors and influx of immune cells. CSF1 plays an important role in immune responses and progression of tumors by participating in recruitment and activation of macrophages. When activated, C3 is cleaved into C3a and C3b. The C3a promotes inflammation, and the C3b binds to the surface of tumor cells and participates in opsonization which is a process by which immune cells eliminate tumor cells.

These experimental results indicate that the composition including the skin keratinocyte-derived ECM as an active ingredient according to the present disclosure exhibits anticancer effects through not only suppressing the proliferation of tumor cells, but also controlling the TME by regulating the expression of genes related to inflammatory response.

### Genes related to ECM

FIG. 4C shows the results of analyzing the changes in the expression of genes related to the ECM in the MDA-MB-231 cells treated with the HaCaT_ECM of Example 1.

As shown in FIG. 4C, when the MDA-MB-231 cells were treated with the HaCaT_ECM, the expression of GDF15, MMP1, ANGPTL4, TFP12, TGFBR3, VASN, PLAT, TIMP3, NTN4, SERPINE2, ANGPTL2, and S100A4 genes, which are genes related to the ECM, was increased by at least two-fold compared to before the HaCaT_ECM treatment.

The genes of which the expression was increased by at least two-fold compared to before the HaCaT_ECM treatment are those related to ECM remodeling. In particular, GDF15 is a gene belonging to the TGF-beta superfamily, whose expression increases during inflammation and stress, thereby contributing to growth and metastasis of tumors, and MMP1 is an enzyme that decomposes collagen, which is a major component of the ECM, and is involved in ECM remodeling and tumor metastasis. ANGPTL4 is involved in angiogenesis, ECM remodeling, and growth and metastasis of tumors, and TFP12 regulates interactions between tumor cells and surrounding cells (fibroblasts and immune cells), thereby participating in ECM remodeling and infiltration of inflammatory and immune cells.

These experimental results indicate that the composition including the skin keratinocyte-derived ECM as an active ingredient according to the present disclosure exhibits anticancer effects through not only suppressing the proliferation of tumor cells, but also controlling the TME by regulating the expression of genes related to ECM remodeling.

### Genes related to cell migration

FIG. 4D shows the results of analyzing the changes in the expression of genes related to cell migration in the MDA-MB-231 cells treated with the HaCaT_ECM of Example 1.

As shown in FIG. 4D, when the MDA-MB-231 cells were treated with the HaCaT-ECM, the expression of CCL2, CSF1, RHOB, IL1B, DNER, ITGA1, CYP1B1, TGFBR3, NRP2, IL6, PLAT, NTN4, CXCL8, ITGA2, JUP, CXCL1, and SAA1 genes, which are genes related to cell migration, was increased by at least two-fold compared to before the HaCaT_ECM treatment.

The genes of which the expression was increased by at least two-fold compared to before the HaCaT_ECM treatment are those related to cell migration, wherein cell migration is closely related to growth, infiltration, and metastasis of tumors. In particular, CCL2 is involved in growth and metastasis of tumors by promoting macrophages and T cell infiltration in the TME, and CSF1 is involved in tumor-associated inflammatory response and immune evasion by regulating the function of macrophages in the TME. RHOB regulates migration and infiltration of tumor cells by modulating interactions between tumor cells and the ECM, IL1B promotes migration of inflammatory cells in the TME and facilitates migration and infiltration of tumor cells, and DNER, a protein associated with the Notch signaling pathway, is involved in migration and invasion of tumor cells.

These experimental results indicate that the composition including the skin keratinocyte-derived ECM as an active ingredient according to the present disclosure exhibits anticancer effects through not only suppressing the proliferation of tumor cells, but also controlling the TME by regulating the expression of genes related to cell migration.

### Genes related to apoptosis

FIG. 4E shows the results of analyzing the changes in the expression of genes related to apoptosis (apoptotic process or cell death) in the MDA-MB-231 cells treated with the HaCaT_ECM of Example 1.

As shown in FIG. 4E, when the MDA-MB-231 cells were treated with the HaCaT_ECM, the expression of TNFSF15, RHOB, IL1B, IL24, LCN2, CYP1B1, ANGPTL4, IL1A, IL6, NFKBIA, SOD2, MAP3K5, IER3, SQSTM1, ZC3H12A, BCL3, and CARD6 genes, which are genes related to apoptosis, was increased by at least two-fold compared to before the HaCaT_ECM treatment.

The genes of which the expression was increased by at least two-fold compared to before the HaCaT_ECM treatment are those related to apoptosis. In particular, TNFSF15 is involved in activation of immune cells and regulation of inflammatory responses, and RHOB is involved in death and migration of tumor cells. IL1B induces apoptosis (cell death) by regulating inflammation and immune responses, IL24 promotes cell death by inducing inflammation, apoptosis, and autophagy, and LCN2 participates in regulating inflammation and immune responses as well as in apoptosis and autophagy. ANGPTL4 affects metabolism and growth of tumor cells by participating in lipid metabolism, and SOD2 is involved in suppressing oxidative stress and protecting cells.

These experimental results indicate that the composition including the skin keratinocyte-derived ECM as an active ingredient according to the present disclosure exhibits anticancer effects through not only suppressing the proliferation of tumor cells, but also controlling the TME by regulating the expression of genes related to apoptosis.

### Genes related to immune checkpoint

FIG. 4F shows the results of analyzing the changes in the expression of genes related to immune checkpoint in the MDA-MB-231 cells treated with the HaCaT_ECM of Example 1.

As shown in FIG. 4F, when the MDA-MB-231 cells were treated with the HaCaT_ECM, the expression of C10ORF54 (VISTA) and DIDO1 genes, which are genes related to immune checkpoint, was increased compared to before the HaCaT_ECM treatment, whereas the expression of CD276 (B7-H3) and CD274 (PD-L1) genes was the same or decreased compared to before the HaCaT_ECM treatment.

C10ORF54 (VISTA), a gene of which the expression was increased compared to before the HaCaT_ECM treatment, is involved in suppressing T cell activation through immunosuppressive function, and DIDO1 creates an immunosuppressive environment by regulating the tryptophan metabolic pathway.

CD276 (B7-H3), a gene showing minimal expression changes compared to before the HaCaT_ECM treatment, suppresses T cell activation and promotes immune evasion of tumors.

CD274 (PD-L1), a gene of which the expression was reduced compared to before the HaCaT_ECM treatment, is expressed in tumor cells and immune cells. PD-1 (PDCD1) is expressed in T cells, and PD-L1/PD-1 is involved in immune evasion.

These experimental results indicate that the composition including the skin keratinocyte-derived ECM as an active ingredient according to the present disclosure exhibits anticancer effects through not only suppressing the proliferation of tumor cells, but also controlling the TME by regulating the expression of genes related immune checkpoint.

### 3.1.3 DAVID - Functional annotation analysis

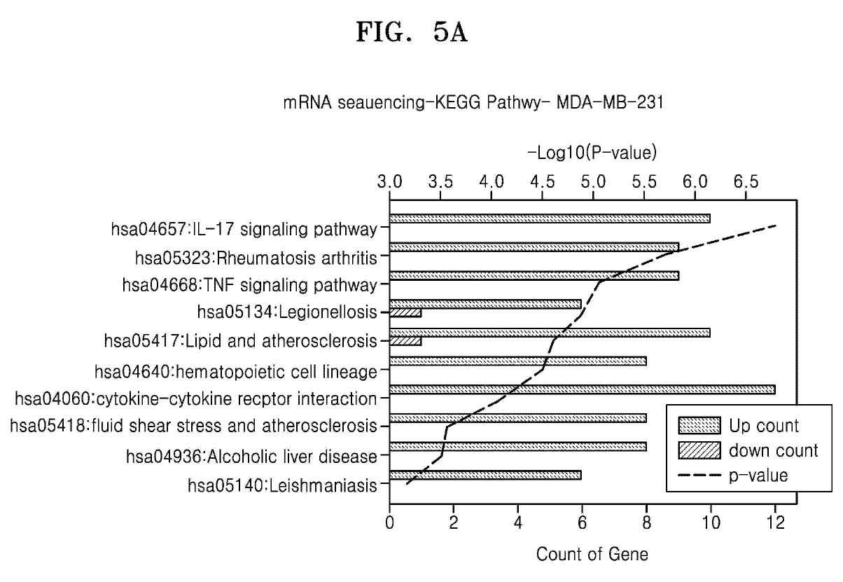

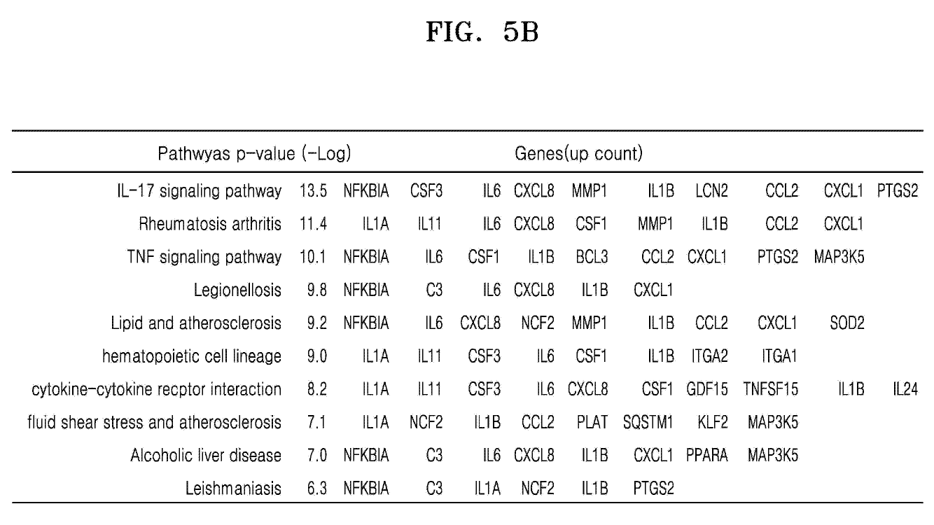

DAVID functional annotation analysis was performed on 162 genes selected at the level of fold change 2, normalized data 4 (log 2), and p-value of 0.05, based on RNA sequencing analysis of the MDA-MB-231 cells treated with the HaCaT_ECM of Example 1, and the results are shown in FIGS. 5A and 5B.

FIGS. 5A and 5B show results of DAVID functional annotation analysis on 162 genes selected at the level of Fold change 2, Normalized Data 4 (Log 2), and p-value of 0.05, based on RNA sequencing analysis of MDA-MB-231 cells treated with HaCaT_ECM. according to an embodiment. Specifically, genes related to the IL-17 signaling pathway, genes related to the TNF signaling pathway, genes related to the hematopoietic cell lineage, and genes related to the cytokine-cytokine receptor interactions are well known to be closely related to the TME. Increased gene expression in the rheumatoid arthritis, legionellosis, and leishmaniasis pathways indicates activation of immune responses. In addition, increased gene expression in the lipid and atherosclerosis, fluid shear stress, and atherosclerosis pathways indicates increased expression of genes related to lipid metabolism, suggesting potential ability to suppress growth of tumor cells or induce apoptosis. The fluid shear stress pathway is associated with cellular stress, and may induce TME changes and apoptosis. Increased gene expression in the alcoholic liver disease pathway indicates that apoptosis may be induced through cellular toxicity rebound and oxidative stress.

As shown in FIGS. 5A and 5B, it was confirmed that, when the MDA-MB-231 cells were treated with the HaCaT_ECM obtained by the method of Example 1, the cells exhibited not only an inhibitory effect on the proliferation of tumor cells within the TME, but also an antitumor effect based on the following: changes in genes related to the IL-17 signaling pathway involved in angiogenesis and immunosuppression; activation of signal transduction related to tumor necrosis and regulation of inflammation and immune responses, upon the increase in NFKB1A, IL6, CSF1, IL1B, CCL2, CXCL1, PTGS2 (COX-2), and MAP3K5 (ASK1) of the TNF signaling pathway; changes in the expression between pro-inflammatory factors and anti-inflammatory factors such as IL1A, IL6, CXCL8, and CSF3 related to the cytokine-cytokine receptor interactions; and regulation of the expression of C10ORF54 (VISTA), DIDO1, CD276 (B7-H3), and CD274 (PD-L1)genes related to the immune checkpoint.

### 3.2 Confirmation of gene expression changes in MCF7 cells

### 3.2.1 Confirmation of gene group with significant expression changes

First, MCF7 cells, a breast cancer cell line, were treated with the HaCaT_ECM according an embodiment at a concentration of 3000 μg/mL and cultured for 24 hours. Total RNA was extracted from the cultures by using the RNeasy Mini kit (Qiagen), and the concentration was measured using NanoVue (GE HealthCare, USA). Subsequently, mRNA sequencing was requested (Ebiogen, Republic of Korea).

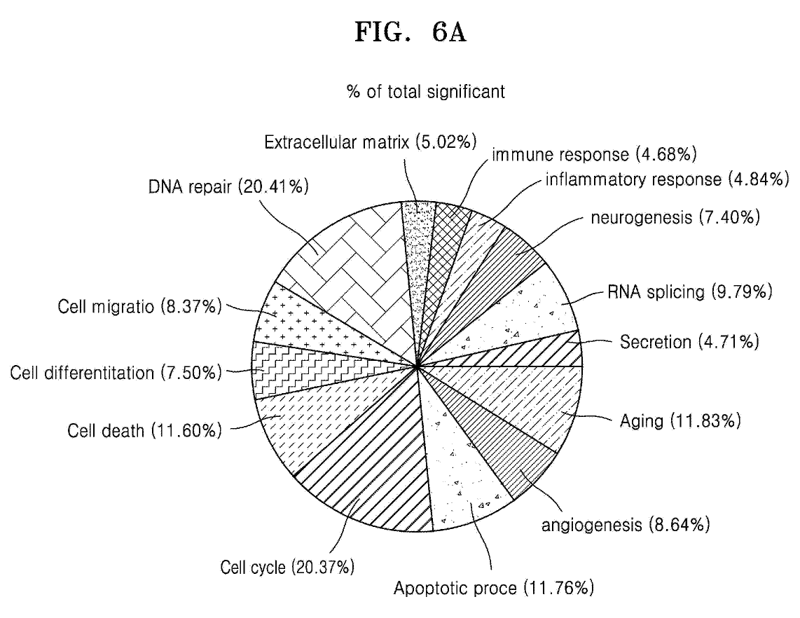

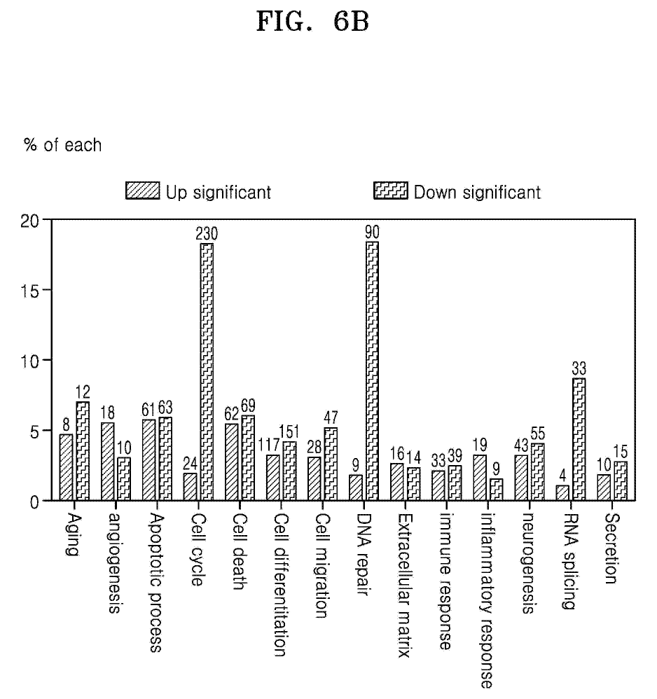

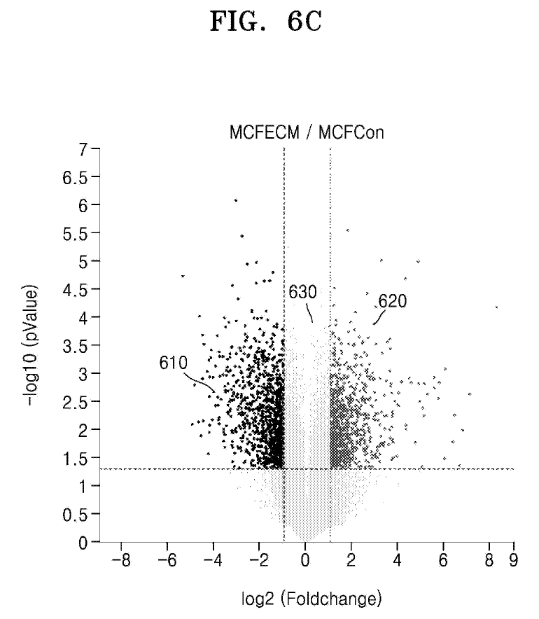

FIG. 6 shows results of RNA sequencing analysis after treating the MCF7 cell lines with the HaCaT_ECM of Example 1, wherein FIG. 6A shows genes that changed by at least two-fold compared to a control group that was not treated with the HaCaT_ECM, as categorized by gene type, FIG. 6B shows proportions of genes that show an increase or a decrease within each gene category, and FIG. 6C shows a scatter plot of genes showing such a two-fold or more change. In FIG. 6, dots 610 indicate a decrease, dots 620 indicate an increase, and dots 630 indicate genes that do not show at least a two-fold difference.

As shown in FIGS. 6A to 6C, it was confirmed that the MCF7 cells treated with the HaCaT_ECM showed significant changes in the expression of genes related to DNA repair, cell cycle, apoptosis (or apoptotic process), cell aging, and RNA splicing (showing at least a two-fold difference in the expression levels compared to before the HaCaT_ECM treatment).

### 3.2.2 Confirmation of expression changes in specific genes

The gene expression changes in MCF7 cells, a breast cancer cell line, treated with the HaCaT_ECM obtained by the method of Example 1 were specifically observed through RNA sequencing analysis.

### Genes related to DNA repair

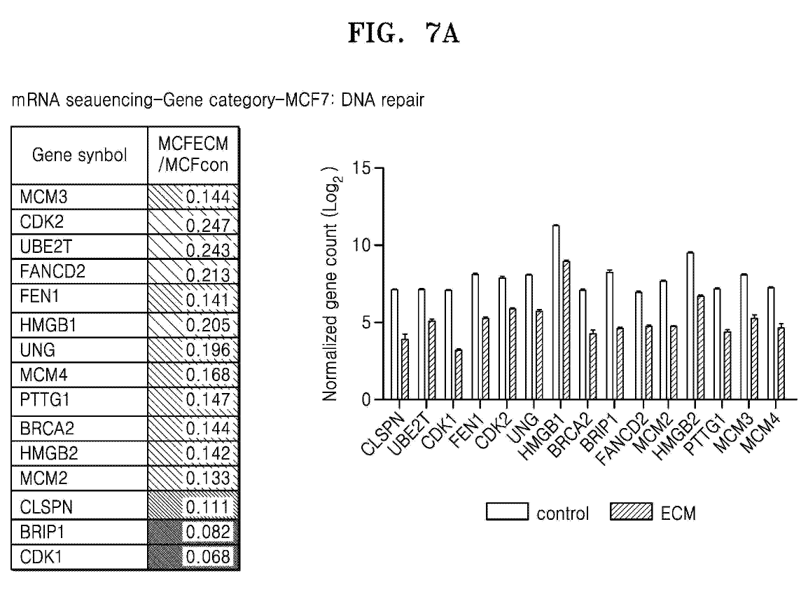

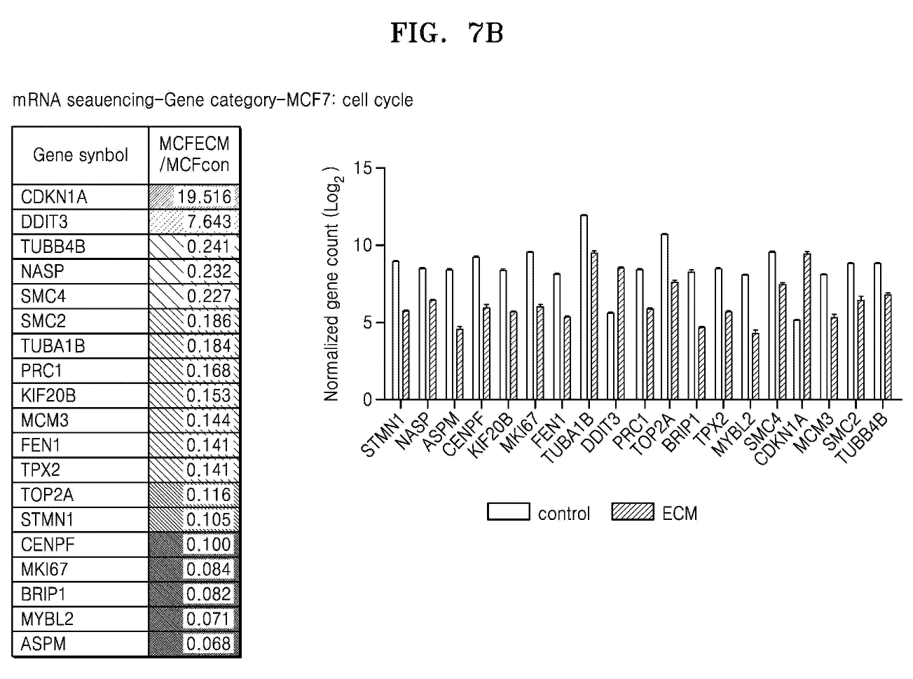

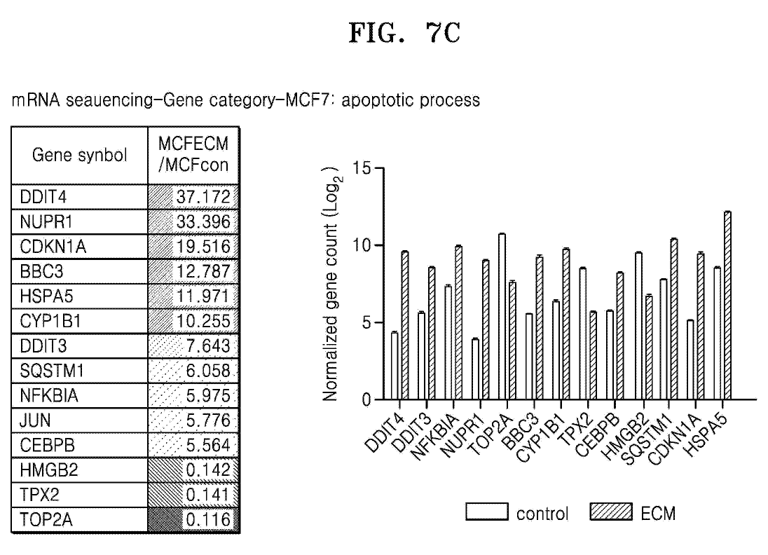

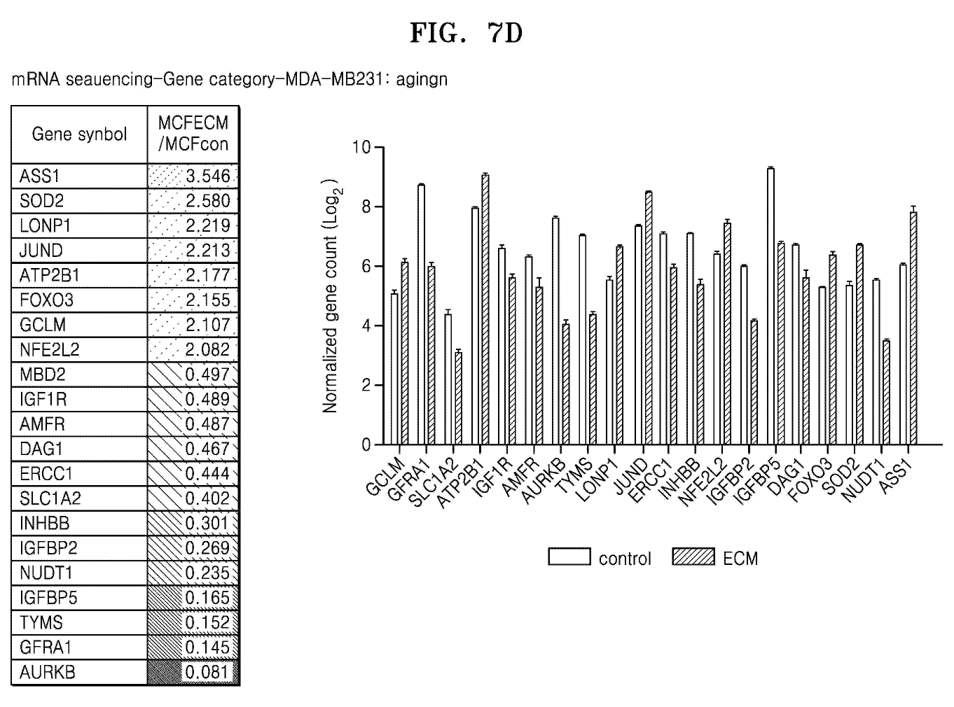

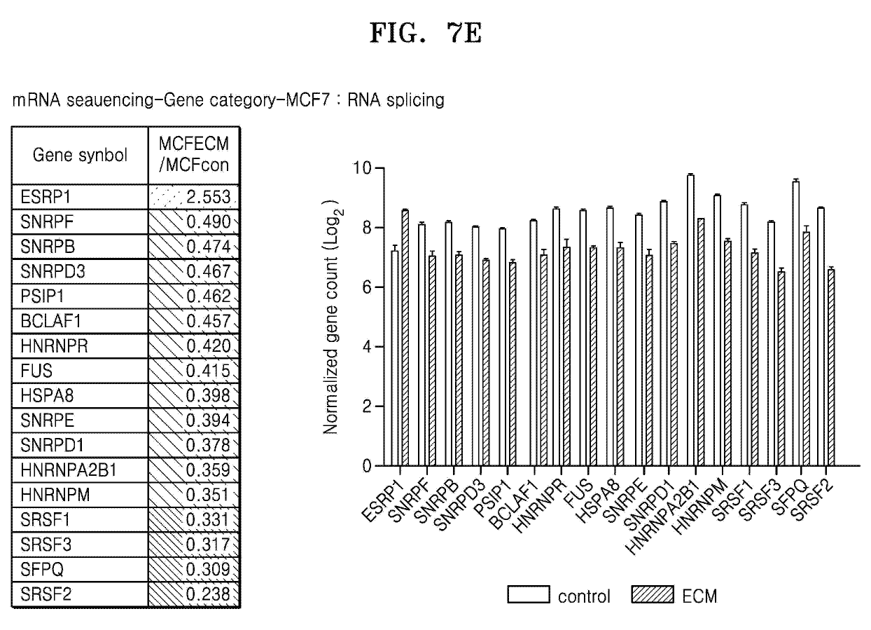

FIG. 7A shows the results of analyzing the changes in the expression of genes related to DNA repair in the MCF7 cells treated with the HaCaT_ECM of Example 1.

As shown in FIG. 7A, the treatment of the MCF7 cells with the HaCaT_ECM resulted in an at least four-fold decrease in the expression of MCM3, FEN1, HMGB1, UNG, MCM4, PTTG1, BRCA2, HMGB2, MCM2, CLSPN, BRIP1, and CDK1 genes, which are genes related to DNA repair, compared to before the HaCaT_ECM treatment. Among the DNA repair-related genes showing an at least four-fold decrease in the expression compared to before the HaCaT_ECM treatment, cyclin-dependent kinase 1 (CDK1) in particular regulates a cell cycle by controlling entry from G2 phase to M phase and cell division. The inhibition of CDK1 expression causes cells to arrest in the G2 phase, allowing time for damaged DNA to be repaired, or promoting apoptosis if repair is impossible. BRCA 1 interacting protein C-terminal helicase 1 (BRIP1) is a protein that interacts with BRCA1 to participate in DNA repair. The inhibition of BRIP1 expression reduces DNA repair ability, promoting apoptosis. Claspin (CLSPN) is a protein that plays an important role in DNA repair checkpoints. When the CLSPN expression is inhibited, the function of the G2/M checkpoint is weakened, the cell cycle is arrested, the genetic instability increases, and apoptosis is induced, thereby suppressing the proliferation of cancer cells.

The decrease in the expression of DNA repair-related genes leads to disruption of the DNA repair process, causing accumulated DNA damage within cells to promote intracellular stress responses and apoptosis, and potentially trigger immune responses within the TME. In addition, the stress responses triggered by DNA damage also affects the expression of inflammatory cytokines and growth factors, thereby affecting changes in the ECM within the TME.

These experimental results indicate that the composition including the skin keratinocyte-derived ECM as an active ingredient according to the present disclosure exhibits anticancer effects through not only suppressing the proliferation of tumor cells, but also regulating the expression of genes related to DNA repair to promote death of cancer cells and controlling the TME.

### Genes related to cell cycle

FIG. 7B shows the results of analyzing the changes in the expression of genes related to cell cycle in the MCF7 cells treated with the HaCaT_ECM of Example 1.

As shown in FIG. 7B, the treatment of the MCF7 cells with the HaCaT_ECM resulted in an at least four-fold decrease in the expression of SMC2, TUBA1B, PRC1, KIP20B, MCM3, FEN1, TPX2, TOP2A, STMN1, CENPF, MKI67, BRIP1, MYBL2, and ASPM genes, which are genes related to cell cycle, compared to before the HaCaT_ECM treatment, and also resulted in an at least seven-fold increase in the expression of DDIT3 and CDKN1A genes compared to before the HaCaT_ECM treatment.

Among the cell cycle-related genes showing an at least four-fold decrease in the expression compared to before the HaCaT_ECM treatment, marker of proliferation Ki-67 (MKI67) in particular is a gene primarily expressed during cell division and serves as a key indicator of cell proliferation. That is, a decrease in the expression of MKI67 indicates a decrease in cell proliferation. BRIP1 is a protein that plays an important role in the DNA damage repair process. When the expression of BRIP1 is decreased, DNA repair efficiency decreases so that cell survival becomes difficult and apoptosis is induced. MYB proto-oncogene like 2 (MYBL2) is a transcription factor important for cell cycle regulation and cell proliferation. When the expression of MYBL2 is decreased, cell cycle progression is suppressed and cell proliferation is inhibited. Abnormal spindle microtubule assembly (ASPM) is a gene involved in cell cycle progression and cell division. When the expression of ASPM is reduced, cell division is suppressed, resulting in a decreased cell proliferation rate.

When the expression of the cell cycle-related genes is decreased by the HaCaT_ECM treatment, the proliferation of cancer cells is suppressed, apoptosis is induced, infiltration of immune cells into the TME is promoted, and an inflammatory response may be induced. In addition, by creating an immune-activated TME, anti-tumor immune responses may be enhanced.

Meanwhile, among the cell cycle-related genes showing an at least seven-fold increase in the expression compared to before the HaCaT_ECM treatment, cyclin-dependent kinase inhibitor 1A (CDKN1A, p21) in particular is a cell cycle inhibitor induced by the p53 tumor suppressor protein. The increased expression of CDKN1A suppresses the activity of the cyclin-CDK complex and blocks cell cycle progression at the G1/S or G2/M checkpoint, thereby inducing apoptosis. DNA damage inducible transcript 3 (DDIT3) is a transcription factor induced by endoplasmic reticulum (ER) stress or various cellular stress situations, and the increased expression of DDIT3 expression induces apoptosis.

When the expression of the cell cycle-related genes is increased by the HaCaT_ECM treatment, antitumor effects that suppress cancer cells may be exhibited through inhibition of each cell cycle and induction of apoptosis. In addition, antigens released from dead cancer cells may promote anti-cancer immune responses that activate immune cells within the TME. The increased expression of stress response genes such as DDIT3 may affect other cells such as fibroblasts and endothelial cells within the TME, thereby suppressing the survival and proliferation of cancer cells within the TME.

These experimental results indicate that the composition including the skin keratinocyte-derived ECM as an active ingredient according to the present disclosure exhibits anticancer effects through not only suppressing the proliferation of tumor cells, but also regulating the expression of the cells related to cell cycle to promote death of cancer cells and controlling the TME.

### Genes related to apoptosis

FIG. 7C shows the results of analyzing the changes in the expression of genes related to apoptosis (or apoptotic process) in the MCF7 cells treated with the HaCaT_ECM of Example 1.

As shown in FIG. 7C, the treatment of the MCF7 cells with the HaCaT_ECM resulted in an at least four-fold decrease in the expression of HMGB2, TPX2, and TOP2A, which are genes related to apoptosis, compared to before the HaCaT_ECM treatment, and also resulted in an at least five-fold increase in the expression of DDIT4, NUPR1, CDKN1A, BBC3, HSPA5, CYP1B1, DDIT3, SQSTM1, JUN, and CEBPB genes compared to before the HaCaT_ECM treatment.

Among the apoptosis-related genes showing an at least four-fold decrease in the expression compared to before the HaCaT_ECM treatment, high mobility group box 2 (HMGB2) in particular regulates the structure of chromatin and the gene expression, and plays an important role in cell growth, differentiation, and DNA damage responses. In this regard, a decrease in the expression of HMGB2 diminishes DNS repair ability, thereby reducing cellular survival ability. Topoisomerase II alpha (TOP2A) plays a role in unwinding the helical structure of DNA during DNA replication and transcription. In this regard, a decrease in the expression of TOP2A leads to improper DNA replication and segregation, thereby causing cell cycle arrest or inducing apoptosis. Targeting protein for Xklp2 (TPX2) is a microtubule-associated protein in cells and is essential for spindle formation. A decrease in the expression of TPX2 causes errors in the cell division process, thereby inducing apoptosis.

When the expression of the apoptosis-related genes is decreased by the HaCaT_ECM treatment, the cell cycle progression and DNA repair ability of cancer cells are impaired, thereby inducing apoptosis. The increased apoptosis of cancer cells may activate immune cells within the TME, thereby enhancing the antitumor immune responses.

Among the apoptosis-related genes showing an at least five-fold increase in the expression compared to before the HaCaT_ECM treatment, DNA damage inducible transcript 4 (DDIT4) in particular increases its expression under cellular stress conditions such as hypoxia or nutrient deprivation, and inhibits the mammalian target of rapamycin complex 1 (mTORC1) pathway, thereby suppressing cell growth and inducing apoptosis. The expression of nuclear protein 1 (NUPR1) is increased under cellular stress conditions, and a sustained increase in the expression of NUPR1 induces apoptosis. Cyclin-dependent kinase inhibitor 1A (CDKN1A) is induced by the p53 tumor suppressor protein, and the increased expression of CDKN1A inhibits the cell cycle and cell proliferation, and promotes the cell aging and apoptosis.

When the expression of the apoptosis-related genes is increased by the HaCaT_ECM treatment, immune cells within the TME, such as dendritic cells and macrophages, may be stimulated, thereby promoting antitumor immune responses.

These experimental results indicate that the composition including the skin keratinocyte-derived ECM as an active ingredient according to the present disclosure exhibits anticancer effects through not only suppressing the proliferation of tumor cells, but also regulating the expression of the genes related to apoptosis to promote death of cancer cells and controlling the TME.

### Genes related to aging

FIG. 7D shows the results of analyzing the changes in the expression of genes related to aging in the MCF7 cells treated with the HaCaT_ECM of Example 1.

As shown in FIG. 7D, the treatment of the MCF7 cells with the HaCaT_ECM resulted in an at least four-fold decrease in the expression of IGFBP5, TYMS, GFRA1, and AURKB genes, which are genes related to aging, compared to before the HaCaT_ECM treatment, and also resulted in an at least two-fold increase in the expression of the expression of ASS1, SOD2, LONP1, JUND, ATP2B1, FOXO3, GCLM, and NFE2L2 genes compared to before the HaCaT_ECM treatment.

Among the aging-related genes showing an at least four-fold decrease in the expression compared to before the HaCaT_ECM treatment, thymidylate synthase (TYMS) in particular is involved in thymidine synthesis during DNA synthesis. DNA synthesis is essential for cell proliferation, and thus a decreased in the expression of leads to inhibition of division and proliferation of cells. GDNF family receptor alpha 1 (GFRA1) interacts with glial cell-derived neurotrophic factor (GDNF) to transmit cell survival signals. GFRA1 is primarily involved in survival and growth of cells in the nervous system. However, it is known that activation of this pathway in some cancer cells may promote survival and proliferation of cancer cells. When the expression of GFRA1 is decreased, cancer cells fail to receive sufficient survival signals, resulting in lower survival rates under stress conditions, thereby promoting apoptosis. Aurora kinase B (AURKB) is involved in alignment and separation processes of chromosomes during mitosis. A decrease in the expression of AURKB suppresses cell division and subsequently inhibits cell proliferation.

When the expression of the aging-related genes is decreased by the HaCaT_ECM treatment, the proliferation of cancer cells is suppressed, promoting immune activation within the TME. Furthermore, the availability of resources that immune cells or fibroblasts may utilize may be increased. That is, a decrease in expression of the aging-related genes by the HaCaT_ECM treatment changes the overall characteristics of the TME, creating a more unfavorable environment for survival of cancer cells.

Meanwhile, among the aging-related genes showing an at least two-fold increase in the expression compared to before the HaCaT_ECM treatment, argininosuccinate synthase 1 (ASS1) is a key enzyme in the arginine biosynthesis pathway. Arginine is essential for protein synthesis, nitrogen metabolism, cell growth and survival, and many cancer cells depend on arginine. An increase in the expression of ASS1 may increase arginine synthesis in cancer cells, but may also cause an imbalance in amino acid metabolism, potentially leading to cellular stress and aging. Superoxide dismutase 2 (SOD2) is an antioxidant enzyme that detoxifies reactive oxygen species (ROS) in mitochondria. It may protect cells from oxidative damage, but may also promote cell aging. Senescent cells exhibit reduced proliferative ability and are also associated with activation of tumor suppression mechanisms. Lon peptidase 1 (LONP1) is a protease that degrades damaged proteins within mitochondria, wherein impaired mitochondrial function may induce cellular aging, which is associated with suppression of cancer cell proliferation.

When the expression of the aging-related genes is increased by the HaCaT_ECM treatment, cells may enable to better withstand environmental stresses such as oxidative stress, but may simultaneously accelerate cell aging. By suppressing proliferation of senescent cancer cells or inducing apoptosis, antitumor effects may be exhibited. In addition, accumulation of senescent cells within the TME may modulates inflammatory responses within the TME, thereby activating anti-tumor immune responses.

These experimental results indicate that the composition including the skin keratinocyte-derived ECM as an active ingredient according to the present disclosure exhibits anticancer effects through not only suppressing the proliferation of tumor cells, but also regulating the expression of the genes related to aging to promote death of cancer cells and controlling the TME.

### Genes related to RNA splicing

FIG. 7E shows the results of analyzing the changes in the expression of genes related to RNA splicing in the MCF7 cells treated with the HaCaT_ECM of Example 1.

As shown in FIG. 7E, the treatment of the MCF7 cells with the HaCaT_ECM resulted in an at least two-fold decrease in the expression of MCF7 cells, the expression of SNRPF, SNRPB, SNRPD3, PSIP1, BCLAF1, HNRNPR, FUS, HSPA8, SNRPE, SNRPD1, HNRNPA2B1, HNRNPM, SRSF1, SRSF3, SFPQ, and SRSF2 genes, which are genes related to RNA splicing, compared to before the HaCaT_ECM treatment, and also resulted in an at least two-fold increase in the expression of ESRP1 gene compared to before the HaCaT_ECM treatment. Among the RNA splicing-related genes showing an at least two-fold decrease in the expression compared to before the HaCaT_ECM treatment, serine and arginine rich splicing factors 1, 2, and 3 (SRSF1, 2, and 3) in particular are involved in the process of removing introns from transcribed RNA and connecting exons to form final mRNA, and have a significant impact on protein expression, function, and various physiological processes of cells. A decrease in the expression of SRSF1, 2, and 3 results in abnormal splicing patterns in cancer cells, leading to abnormal protein expression and dysfunction in tumor cells, thereby exhibiting antitumor effects.

Heterogeneous nuclear ribonucleoprotein M (HNRNPM) is an RNA-binding protein that is involved in splicing as well as RNA stability and translation regulation. A decrease in the expression of HNRNPM affects growth, differentiation, and apoptosis of cancer cells, thereby suppressing tumor growth.

When the expression of the RNA splicing-related genes is decreased by the HaCaT_ECM treatment, immune cells within the TME may be activated, thereby exhibiting an anti-tumor effect.

Meanwhile, among the RNA splicing-related genes showing an at least two-fold increase in the expression compared to the HaCaT_ECM treatment, epithelial splicing regulatory protein 1 (ESRP1) in particular maintains epithelial cell-specific splicing patterns in epithelial cells, and plays a role in regulating an epithelial and epithelial-mesenchymal transition (EMT) process of the cells. When an increase in the expression of ESRP1 induces maintenance of the characteristics of the epithelial cells, the EMT is suppressed, thereby reducing the metastatic ability of cancer cells.

ESRP1 influences cell-to-cell interactions and interactions with the ECM, and plays an important role in maintaining the characteristics of the epithelial cell. An increase in the expression of ESRP1 enhances cell-to-cell adhesion and interactions, thereby reducing tumor metastasis and invasion within the TME. In addition, ESRP1 is involved in maintaining epithelial maintenance, creating an environment within the TME where immune cells may better recognize and attack tumor cells.

These experimental results indicate that the composition including the skin keratinocyte-derived ECM as an active ingredient according to the present disclosure exhibits anticancer effects through not only suppressing the proliferation of tumor cells, but also regulating the expression of the genes related to RNA splicing to promote death of cancer cells and controlling the TME.

### 3.2.3 DAVID - Functional annotation analysis

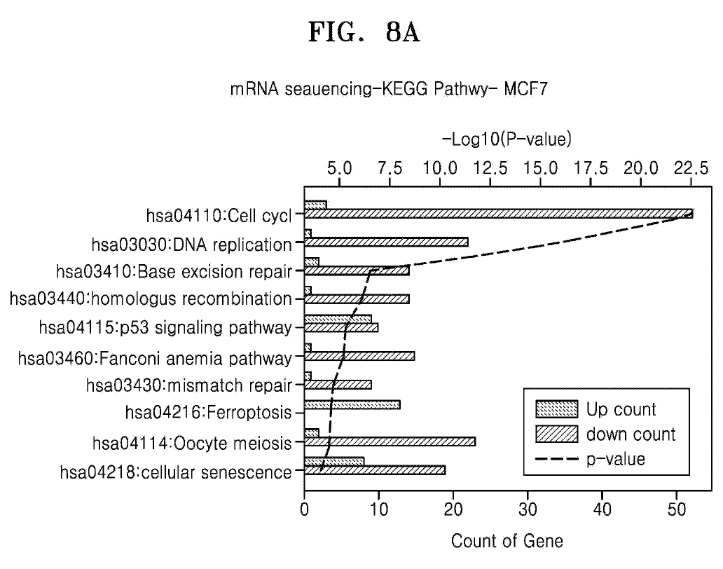

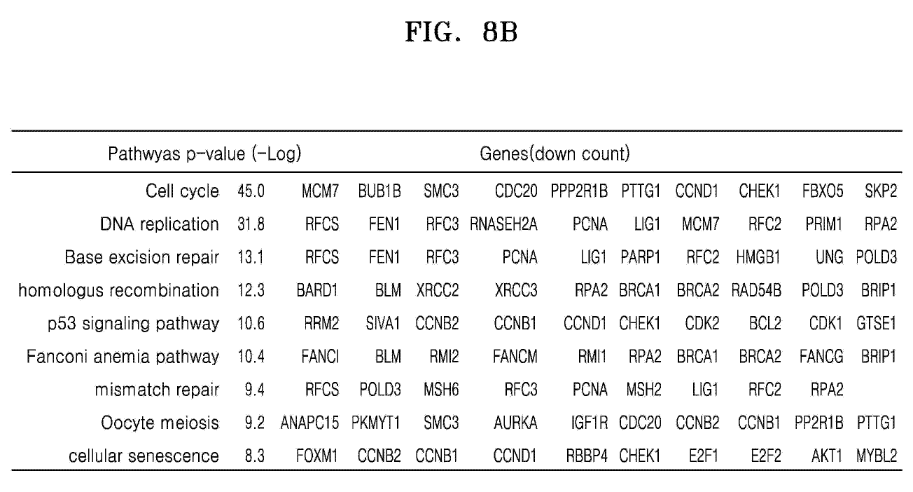

DAVID functional annotation analysis was performed on 1403 genes selected at the level of fold change 2, normalized data 4 (log 2), and p-value of 0.05, based on RNA sequencing analysis of the MCF7 cells treated with the HaCaT_ECM of Example 1, and the results are shown in FIGS. 8A and 8B.

FIGS. 8A and 8B show the results of DAVID functional annotation analysis on 1403 genes selected at the level of fold change 2, normalized data 4 (Log 2), and p-value of 0.05 based on RNA sequence analysis of MCF7 cells treated with the HaCaT_ECM of Example 1. Specifically, the inhibition of the expression of genes involved in the cell cycle, DNA replication, and p53 signaling pathway inhibits division and growth of cells, inhibits DNA replication, and increases apoptosis, thereby activating immune cells within the TME. Therefore, by controlling inhibition of the expression of genes involved in the cell cycle, DNA replication, and p53 signaling pathway, antitumor immune responses may be induced and mobility and invasiveness of tumor cells may be reduced. A decrease in the expression of genes involved in DNA repair pathways, such as base excision repair, homologous recombination, and mismatch repair, may cause cells to proliferate with damaged DNA, inducing death of cancer cells and potentially increasing sensitivity to anticancer treatments. Genes involved in the Fanconi anemia pathway play an important role in repairing DNA cross-link damage. A decrease in the expression of genes involved in the Fanconi anemia pathway induces death of tumor cells due to accumulation of DNA damage, thereby inducing antitumor immune responses within the TME. A decrease in the expression of genes involved in oocyte meiosis affects division and growth of cells. A decrease in the expression of genes involved in cellular senescence is associated with aging and apoptosis of tumor cells.

As shown in FIGS. 8A and 8B, the treatment of the MCF7 cells with the HaCaT_ECM of resulted in: a decrease in the expression of CCDN1, CHEK1, and FBXO5 genes involved in the cell cycle pathway, thereby suppressing the cell cycle and subsequently suppressing the proliferative capacity of tumor cells; a decrease in the expression of RFC5, FEN1, RFC3, PCNA, LIG1, MCM7, RFC2, PRIM1, and RPA2 genes involved in the DNA replication pathway, thereby causing a decrease in proliferation of tumor cells; and a decrease in the expression of FOXM1, CCNB2, CCDN1, RNBBP4, CHEK1, and E2F1 genes involved in cellular senescence, thereby regulating the expression of genes involved in the cell senescence, tumor suppression, and p53 signal pathways, thereby exhibiting an anti-tumor effect.

### Experimental Example 4. Confirmation of anticancer effects following HaCaT_ECM administration in vivo

To confirm the anticancer effects of the HaCaT_ECM treatment in vivo, tumors were created by injecting MDA-MB231 cells into mice, and the decellularized ECM derived from skin keratinocytes (HaCaT_ECM) obtained by the method of Example 1 was administered. The results were observed as follows.

Specifically, MDA-MB231 cells were injected subcutaneously at a density of 2×10⁷ cell/200 μL into the axillary region of a 6-week-old female Balb/c nude mouse. When the tumor size reached an average of 100 mm², the HaCaT_ECM was dissolved in PBS and prepared at 1% and 3% concentrations, and then administered intratumorally once a week for 3 weeks.

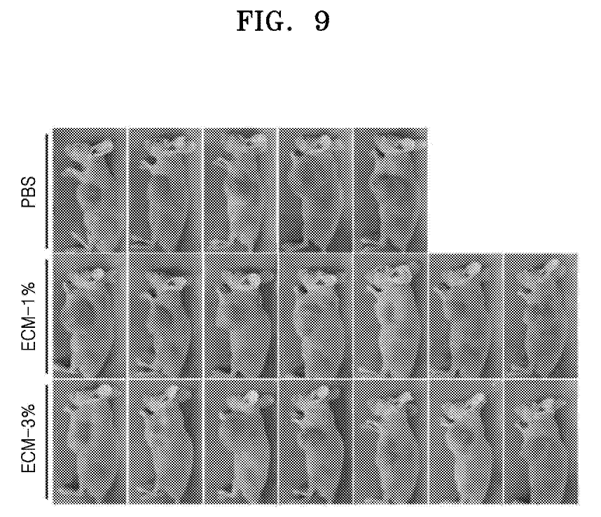

FIG. 9 shows photographs of the mouse tumor 3 weeks after administration of the HaCaT_ECM according to an embodiment.

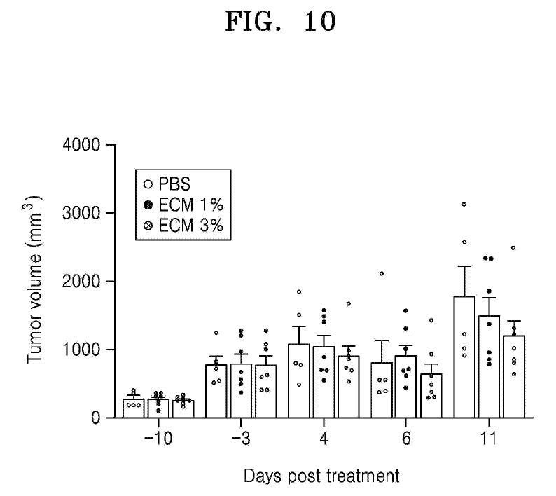

FIG. 10 shows a graph measuring changes in the tumor size resulting from administration of the HaCaT_ECM according to an embodiment.

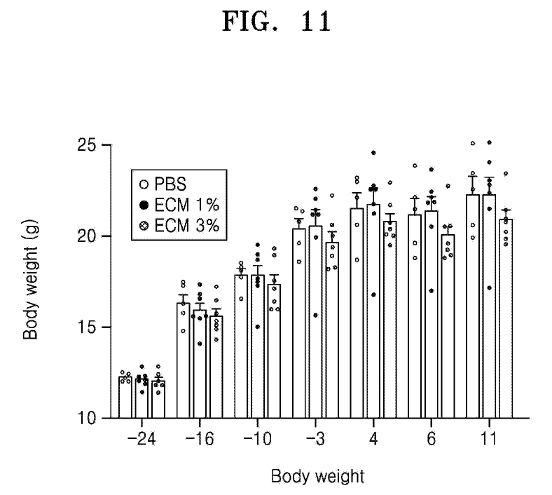

FIG. 11 shows a graph measuring changes in the mouse body weight following administration of the HaCaT_ECM according to an embodiment.

As shown in FIGS. 9 to 11, it was confirmed that administration of the HaCaT_ECM suppressed an increase in the tumor size.

These experimental results indicate that the composition including the skin keratinocyte-derived ECM as an active ingredient according to the present disclosure exhibits an antitumor effect in vivo.

Although the present disclosure has been described with reference to the preferred embodiments mentioned above, various modifications and variations are possible without departing from the spirit and scope of the disclosure. In addition, the appended claims include such modifications or variations as fall within the spirit of the present disclosure.

What is claimed is:

1. A method of treating cancer, comprising administering, to a subject in need thereof, a composition comprising an effective amount of a skin keratinocyte-derived extracellular matrix (ECM) as an effective ingredient.

2. A method of suppressing angiogenesis, comprising administering, to a subject in need thereof, a composition comprising an effective amount of a skin keratinocyte-derived extracellular matrix (ECM) as an effective ingredient.

3. A method of suppressing metastasis, comprising administering, to a subject in need thereof, a composition comprising an effective amount of a skin keratinocyte-derived extracellular matrix (ECM) as an effective ingredient.

4. The method of claim 1, wherein the ECM is obtained by culturing skin keratinocytes and subsequently decellularizing the skin keratinocytes.

5. The method of claim 1, wherein the cancer is at least one selected from oral cancer, liver cancer, stomach cancer, colon cancer, breast cancer, lung cancer, non-small cell lung cancer, bronchial cancer, nasopharyngeal cancer, laryngeal cancer, bone cancer, non-small cell bone cancer, pancreatic cancer, skin cancer (melanoma, etc.), basal cell carcinoma, head cancer, neck cancer, skin cancer, cervical cancer, ovarian cancer, ovarian germ cell tumor, colorectal cancer, small bowel cancer, rectal cancer, fallopian tube cancer, anal cancer, endometrial cancer, vaginal carcinoma, vulvar carcinoma, Hodgkin's disease, salivary gland cancer, tongue cancer, esophageal cancer, duodenal cancer, lymphoma, bladder cancer, gallbladder cancer, endocrine cancer, thyroid cancer, parathyroid cancer, adrenal cancer, sarcoma, pseudomyxoma peritonei, urethral cancer, ureteral cancer, penile cancer, testicular cancer, prostate cancer, chronic leukemia, acute leukemia, multiple myeloma, lymphocytic lymphoma, pediatric lymphoma, renal cancer, renal pelvis cancer, ureter cancer, renal cell carcinoma, renal pelvis carcinoma, vulvar cancer, thymus cancer, central nervous system tumor, primary central nervous system lymphoma, neuroblastoma, astrocytoma, meningioma, choroidal melanoma, ampulla of Vater cancer, peritoneal cancer, small cell carcinoma, spinal cord tumor, brain stem glioma, and pituitary adenoma.

6. The method of claim 1, wherein the composition is administered intratumorally.

7. The method of claim 1, wherein the composition inhibits proliferation of cancer cells.

8. The method of claim 1, wherein the composition controls a tumormicroenvironment (TME).

9. The method of claim 8, wherein controlling the TME is suppressing at least one selected from the group consisting of angiogenesis, inflammatory response, cell migration, and immunosuppressive action, within the TME.

10. The method of claim 1, wherein the composition comprises 0.001 to 10.0 (w/v)% of decellularized ECM.

11. The method of claim 1, wherein the composition is administered in combination with other anticancer drugs.

12. The method of claim 11, wherein the other anticancer drugs comprise a standard anticancer therapeutic agent, an immune checkpoint inhibitor, or an immunotherapy.
